(19) United States
(12) Patent Application Publication
(10) Pub. No.: US 2018/0327723 A1
(43) Pub. Date: Nov. 15, 2018

(54) Production of Glycosylated Nootkatol in Recombinant Hosts

(71) Applicant: Evolva SA, Reinach (CH)

(72) Inventors: Dayal Saran, Lexington, KY (US); Jorgen Hansen, Frederiksberg (DK); Philippe Prochasson, Georgetown, KY (US); Patrick Roussel, Mulhouse (FR); Laura Occhipinti, Zurich (CH); Alexei Ouspenski, Mulhouse (FR); Sumire Honda Malca, Loerrach (DE)

(21) Appl. No.: 15/774,134

(22) PCT Filed: Nov. 14, 2016
(86) PCT No.: PCT/EP2016/077614

§ 371 Date: May 7, 2018

Related U.S. Application Data
(60) Provisional application No. 62/255,578, filed on Nov. 16, 2015.

(51) Int. Cl. C12N 9/10; C12P 19/44

(52) U.S. Cl. CPC C12N 9/1048; C12P 19/44; C12N 15/70; C12Y 402/03073; C12Y 106/02004

(57) ABSTRACT

The invention relates to methods for producing glycosylated nootkatol. In particular, a recombinant host comprising a gene encoding a UDP-glycosyltransferase polypeptide capable of glycosylating nootkatol is disclosed. Glycosylation of nootkatol detoxifies nootkatol, allowing for greater production of (glycosylated-)nootkatol, a precursor of nootkatone, and therefore greater production of nootkatone. The invention also relates to methods of converting non-toxic, glycosylated nootkatol produced by a recombinant host to nootkatol, wherein the nootkatol can subsequently be converted to large quantities of nootkatone to be used in flavorings, perfumes, and/or insect repellents.

### BACKGROUND OF THE INVENTION

### Field of the Invention

The present invention relates to methods and materials for the biosynthesis of glycosylated nootkatol in recombinant hosts. The present invention also relates to methods of reducing nootkatol-mediated cellular toxicity by glycosylation of nootkatol, thereby allowing for production of large quantities of nootkatone. More particularly, the present invention relates to conversion of glycosylated nootkatol to nootkatone for use in flavoring, perfume, and insect repellent applications.

### Description of Related Art

Valencene (1,2,3,5,6,7,8,8a-octahydro-7-isopropenyl-1,8a-dimethyl-naphthalene) and nootkatone (4,4a,5,6,7,8-hexahydro-6-isopropenyl-4,4a-dimethyl-2(3H)-napthalenone) are sesquiterpenes that occur in essential oils, such as citrus fruits, including orange and grapefruit. Valencene is produced by cyclization of the acyclic pyrophosphate terpene precursor, farnesyl diphosphate (FPP), and oxidation of valencene results in the formation of nootkatone. Valencene and nootkatone are both used in the perfume and flavor industry. Alternatively, nootkatone may be used as an insecticide (see, e.g., WO 2014/150599, which has been incorporated by reference herein in its entirety). Methods to purify sesquiterpenes, such as valencene and nootkatone, from citrus fruits are known in the art. See, e.g., U.S. Pat. No. 4,693,905, U.S. Pat. No. 4,973,485, U.S. Pat. No. 6,495,193, and U.S. 2003/0185956, each of which has been incorporated by reference herein in its entirety. However, since nootkatone is present in trace amounts in plants, chemical synthesis, which involves use of hazardous oxidizing agents, has been used commercially to produce nootkatone from valencene.

Nootkatol (2,3,4,4a,5,6,7,8-octahydro-6-isopropenyl-4,4a-dimethyl-2-naphtalenol) is also produced from the oxidation of valencene and has been shown to be a precursor to nootkatone. See, e.g., U.S. Pat. No. 5,847,226 and GB 1299299, each of which is incorporated by reference herein in its entirety. Co-expression of a cytochrome P450, cytochrome P450 reductase, and a valencene synthase in yeast has been shown to produce (+)-nootkatone and several products including trans-nootkatol and cis-nootkatol. See, Cankar et al., 2011, FEBS Lett. 585(1):178-82. However, as shown in Gavira et al., 2013, Metab Eng. 18:25-35, low nootkatone yields in yeast were found to be due, in part, to cellular toxicity to nootkatol and nootkatone and accumulation of nootkatol in yeast cell hydrophobic endomembranes. Therefore, the toxic effects of both nootkatol and nootkatone are a significant hindrance to cellular production of nootkatol and nootkatone.

Although nootkatone is valuable for a wide variety of applications, including flavorings, perfumes, and insect repellents, chemical production of nootkatone has proven to be labor intensive, expensive, and hazardous and recombinant production of nootkatone has thus far proven to be inefficient due to cellular toxicity to nootkatol and nootkatone. Thus, there remains a need for production of high yields of nootkatone for commercial uses.

### SUMMARY OF THE INVENTION

It is against the above background that the present invention provides certain advantages and advancements over the prior art.

Although this invention disclosed herein is not limited to specific advantages or functionalities, the invention provides a recombinant host comprising one or more of:
(a) a gene encoding a valencene synthase polypeptide;
(b) a gene encoding a cytochrome P450 hydroxylase polypeptide;
(c) a gene encoding a cytochrome P450 reductase polypeptide; and/or
(d) a gene encoding a glycosyltransferase (UGT) polypeptide, wherein the UGT polypeptide is capable of glycosylating nootkatol;

wherein at least one of said genes is a recombinant gene; wherein the recombinant host produces glycosylated nootkatol.

In one aspect of the recombinant host disclosed herein,
(a) the valencene synthase polypeptide comprises a valencene synthase polypeptide having at least 50% identity to an amino acid sequence set forth in SEQ ID NO:20;
(b) the cytochrome P450 hydroxylase polypeptide comprises a cytochrome P450 hydroxylase polypeptide having at least 50% identity to an amino acid sequence set forth in SEQ ID NO:2 or SEQ ID NO:4;
(c) the cytochrome P450 reductase polypeptide comprises a cytochrome P450 reductase polypeptide having at least 50% identity to an amino acid sequence set forth in SEQ ID NO:6 or SEQ ID NO:8; and/or
(d) the UGT polypeptide comprises a UGT polypeptide having at least 50% identity to an amino acid sequence set forth in SEQ ID NO:10, SEQ ID NO:12, SEQ ID NO:14, SEQ ID NO:16, OR SEQ ID NO:18.

In another aspect of the recombinant host disclosed herein, the glycosylated nootkatol comprises monoglycosylated, diglycosylated, triglycosylated, or polyglycosylated nootkatol.

In another aspect of the recombinant host disclosed herein, the recombinant host is characterized by a relative colony-forming unit (CFU) value of at least 0.9.

In another aspect of the recombinant host disclosed herein, the glycosylated nootkatol produced is not toxic to the recombinant host.

In another aspect of the recombinant host disclosed herein, the host further comprises a downregulated, deleted or functionally disrupted endogenous gene encoding an enzyme capable of cleaving a saccharide from glycosylated nootkatol.

The invention further provides a method of producing glycosylated nootkatol, comprising:
(a) growing a recombinant host disclosed herein in a culture medium;

wherein the glycosylated nootkatol is synthesized by the recombinant host; and
(b) optionally isolating the glycosylated nootkatol.

The invention further provides a method for producing glycosylated nootkatol from a bioconversion reaction, comprising:
(a) growing a recombinant host in a culture medium;
   wherein the host comprises a gene encoding a UGT polypeptide capable of in vivo glycosylation of nootkatol and optionally functionally disrupting an endogenous gene encoding an enzyme capable of cleaving a saccharide from glycosylated nootkatol; wherein the gene encoding the UGT polypeptide is expressed in the recombinant host;
(b) contacting the recombinant host with nootkatol in a reaction buffer to produce glycosylated nootkatol; and
(c) optionally isolating the glycosylated nootkatol.

In one aspect of the method disclosed herein, the UGT polypeptide comprises a UGT polypeptide having at least 50% identity to an amino acid sequence set forth in SEQ ID NO:10, SEQ ID NO:12, SEQ ID NO:14, SEQ ID NO:16, OR SEQ ID NO:18.

In another aspect of the method disclosed herein, the method further comprises a step of cleavage of sugar moieties of the glycosylated nootkatol, wherein nootkatol can be isolated from the culture medium.

In one aspect, the step of cleavage of the sugar moieties of the glycosylated nootkatol comprises enzymatic cleavage.

In one aspect, enzymatic cleavage comprises treating the culture medium with an enzyme capable of cleaving sugar moieties.

In another aspect of the method disclosed herein, the enzyme comprises β-glucosidase, cellulase, cellobiase, β-galactosidase, β-glucuronidase, or EXG1.

In another aspect of the method disclosed herein, the step of cleavage of the sugar moieties of the glycosylated nootkatol comprises chemical cleavage.

In one aspect, chemical cleavage comprises treating the culture medium with a weak acid or under other conditions capable of cleaving sugar moieties.

In one aspect, the weak acid comprises an organic acid or an inorganic acid.

In some aspects, the recombinant hosts disclosed herein comprises a plant cell, a mammalian cell, an insect cell, a fungal cell, or a bacterial cell.

In one aspect, the bacterial cell comprises Escherichia bacteria cells, for example, Escherichia coli cells; Lactobacillus bacteria cells; Lactococcus bacteria cells; Cornebacterium bacteria cells; Acetobacter bacteria cells; Acinetobacter bacteria cells; or Pseudomonas bacterial cells.

In one aspect, the fungal cell comprises a yeast cell.

In one aspect, the yeast cell is a cell from Saccharomyces cerevisiae, Schizosaccharomyces pombe, Yarrowia lipolytica, Candida glabrata, Ashbya gossypii, Cyberlindnera jadinii, Pichia pastoris, Kluyveromyces lactic, Hansenula polymorpha, Candida boidinii, Arxula adeninivorans, Xanthophyllomyces dendrorhous, or Candida albicans species.

In one aspect, the yeast cell is a Saccharomycete.

In one aspect, the yeast cell is a cell from the Saccharomyces cerevisiae species.

In one aspect, the yeast cell comprises a downregulated, deleted or functionally disrupted EXG1.

The invention further provides a method for producing glycosylated nootkatol from an in vitro reaction comprising contacting nootkatol with one or more UGT polypeptides in the presence of one or more UDP-sugars.

In one aspect of the method disclosed herein, the UGT polypeptide comprises a UGT polypeptide having at least 50% identity to an amino acid sequence set forth in SEQ ID NO:10, SEQ ID NO:12, SEQ ID NO:14, SEQ ID NO:16, OR SEQ ID NO:18.

In one aspect of the method disclosed herein, the one or more UDP-sugars comprise UDP-glucose, UDP-rhamnose, or UDP-xylose.

In one aspect of the method disclosed herein, the nootkatol comprises plant-derived or synthetic nootkatol.

In another aspect, a method disclosed herein further comprises a step of converting nootkatol to nootkatone.

In another aspect of a method disclosed herein, the step of converting nootkatol to nootkatone comprises chemical or biocatalytic conversion of nootkatol to nootkatone.

In some aspects, a method disclosed herein further comprises a step of detecting the isolated glycosylated nootkatol, nootkatol, and/or nootkatone by thin layer chromatography (TLC), high-performance liquid chromatography (HPLC), ultraviolet-visible spectroscopy/spectrophotometry (UV-Vis), liquid chromatography-mass spectrometry (LC-MS), or nuclear magnetic resonance (NMR).

The invention further provides a glycosylated nootkatol composition produced by a recombinant host and/or method disclosed herein.

The invention further provides a nootkatol composition produced by a method disclosed herein.

The invention further provides a nootkatone composition produced by a method disclosed herein.

In some aspects of the nootkatone composition disclosed herein, the nootkatone composition is used in a flavoring, a perfume, and/or an insect repellent.

These and other features and advantages of the present invention will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE DRAWINGS

The following detailed description of the embodiments of the present invention can be best understood when read in conjunction with the following drawings, where like structure is indicated with like reference numerals and in which:

FIG. 1A shows the chemical structure of nootkatol, and FIG. 1B shows the basic chemical structure of glycosylated nootkatol. The “saccharide” moiety of glycosylated nootkatol can be a mono-, di-, tri-, or polysaccharide.

FIG. 2 is a schematic showing a pathway for production of glycosylated nootkatol, as disclosed herein.

FIG. 3 shows viability (in relative colony-forming units; CFU) of S. cerevisiae cells treated with 0.0, 0.06, 0.125, or 0.5 g/L nootkatol or glycosylated nootkatol for 5 h and subsequently plated. See Example 2.

FIG. 4 shows nootkatol production (in mg/L) in S. cerevisiae strains comprising an Eryngium glaciale valence synthase (SEQ ID NO:19, SEQ ID NO:20) and either i) Hyoscyamus muticus P450 (SEQ ID NO:1, SEQ ID NO:2) and Nicotiana cytochrome P450 reductase (SEQ ID NO:5, SEQ ID NO:6) or ii) Chicorium intybus cytochrome p450 hydroxylase (SEQ ID NO:3, SEQ ID NO:4) and Arabidopsis thaliana cytochrome p450 reductase (SEQ ID NO:7, SEQ ID NO:8). See Example 3.

FIG. 5 shows glycosylated nootkatol production (in mg/L) in S. cerevisiae strains comprising Eryngium glaciale valence synthase (SEQ ID NO:19, SEQ ID NO:20), Chicorium intybus cytochrome p450 hydroxylase (SEQ ID NO:3, SEQ ID NO:4), Arabidopsis thaliana cytochrome p450 reductase (SEQ ID NO:7, SEQ ID NO:8), and a UDP-glycosyltransferase (UGT) selected from UGT85A1 (SEQ ID NO:9, SEQ ID NO:10), UGT76E1 (SEQ ID NO:11, SEQ ID NO:12), or UGT73E1 (SEQ ID NO:13, SEQ ID NO:14). See Example 3.

FIG. 6 (a-d) shows an LC-MS analysis of Gly-nootkatol standard, with composition confirmed using NMR, allowing subsequent identification of the indicated LC-MS peaks corresponding to the substrate of reactions performed in Example 4 (as exemplified by the LC-MS analysis shown in FIG. 7).

FIG. 7 (a-d) illustrates an example of a successful de-glycosylation experiment as performed in Example 4 and shows the LC-MS analysis of post reaction sample 1 (Depot-40). 7a shows the chromatogram with selected ion monitoring of the Gly-nootkatol m/z, 7b shows the mass spectrum (negative mode) at 3.574 min (Gly-nootkatol elution time), 7c shows the selected ion monitoring of the nootkatol m/z. The selected ion monitoring at m/z 203.514 also gives a signal at the retention of Gly-nootkatol, which is caused by in source cleavage of Gly-nootkatol to nootkatol in the electrospray. FIG. 7d shows the mass spectrum (positive mode) at 4.587 min (nootkatol elution time). Collectively, these figures demonstrate the generation of nootkatol.

FIG. 8 details the structural diagrams, molecular formulae and isotopic masses of the substrates and products identified in the reactions of Example 4.

### DETAILED DESCRIPTION OF THE INVENTION

All publications, patents and patent applications cited herein are hereby expressly incorporated by reference for all purposes.

Before describing the present invention in detail, a number of terms will be defined. As used herein, the singular forms “a,” “an,” and “the” include plural referents unless the context clearly dictates otherwise. For example, reference to a “nucleic acid” means one or more nucleic acids.

It is noted that terms like “preferably,” “commonly,” and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

For the purposes of describing and defining the present invention, it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

Methods well known to those skilled in the art can be used to construct genetic expression constructs and recombinant cells according to this invention. These methods include in vitro recombinant DNA techniques, synthetic techniques, in vivo recombination techniques, and polymerase chain reaction (PCR) techniques. See, for example, techniques as described in Green & Sambrook, 2012, MOLECULAR CLONING: A LABORATORY MANUAL, Fourth Edition, Cold Spring Harbor Laboratory, New York; Ausubel et al., 1989, CURRENT PROTOCOLS IN MOLECULAR BIOLOGY, Greene Publishing Associates and Wiley Interscience, New York, and PCR Protocols: A Guide to Methods and Applications (Innis et al., 1990, Academic Press, San Diego, Calif.).

As used herein, the terms “polynucleotide,” “nucleotide,” “oligonucleotide,” and “nucleic acid” can be used interchangeably to refer to nucleic acid comprising DNA, RNA, derivatives thereof, or combinations thereof.

As used herein, the terms “microorganism,” “microorganism host,” “microorganism host cell,” “recombinant host,” and “recombinant host cell” can be used interchangeably. As used herein, the term “recombinant host” is intended to refer to a host, the genome of which has been augmented by at least one DNA sequence. Such DNA sequences include but are not limited to genes that are not naturally present, DNA sequences that are not normally transcribed into RNA or translated into a protein (“expressed”), and other genes or DNA sequences which one desires to introduce into the non-recombinant host. It will be appreciated that typically the genome of a recombinant host described herein is augmented through stable introduction of one or more recombinant genes. Generally, introduced DNA is not originally resident in the host that is the recipient of the DNA, but it is within the scope of this disclosure to isolate a DNA segment from a given host, and to subsequently introduce one or more additional copies of that DNA into the same host, e.g., to enhance production of the product of a gene or alter the expression pattern of a gene. In some instances, the introduced DNA will modify or even replace an endogenous gene or DNA sequence by, e.g., homologous recombination or site-directed mutagenesis. Suitable recombinant hosts include microorganisms.

As used herein, the term “recombinant gene” refers to a gene or DNA sequence that is introduced into a recipient host, regardless of whether the same or a similar gene or DNA sequence may already be present in such a host. “Introduced,” or “augmented” in this context, is known in the art to mean introduced or augmented by the hand of man. Thus, a recombinant gene can be a DNA sequence from another species, or can be a DNA sequence that originated from or is present in the same species, but has been incorporated into a host by recombinant methods to form a recombinant host. It will be appreciated that a recombinant gene that is introduced into a host can be identical to a DNA sequence that is normally present in the host being transformed, and is introduced to provide one or more additional copies of the DNA to thereby permit overexpression or modified expression of the gene product of that DNA. Said recombinant genes are particularly encoded by cDNA.

As used herein, the terms “codon optimization” and “codon optimized” refer to a technique to maximize protein expression in fast-growing microorganisms such as E. coli or S. cerevisiae by increasing the translation efficiency of a particular gene. Codon optimization can be achieved, for example, by transforming nucleotide sequences of one species into the genetic sequence of a different species. Optimal codons help to achieve faster translation rates and high accuracy. As a result of these factors, translational selection is expected to be stronger in highly expressed genes.

As used herein, the term “engineered biosynthetic pathway” refers to a biosynthetic pathway that occurs in a recombinant host, as described herein, and does not naturally occur in the host.

As used herein, the term “endogenous” gene refers to a gene that originates from and is produced or synthesized within a particular organism, tissue, or cell.

As used herein, the terms “heterologous sequence” and “heterologous coding sequence” are used to describe a sequence derived from a species other than the recombinant host. In some embodiments, the recombinant host is an S. cerevisiae cell, and a heterologous sequence is derived from an organism other than S. cerevisiae. A heterologous coding sequence, for example, can be from a prokaryotic microorganism, a eukaryotic microorganism, a plant, an animal, an insect, or a fungus different than the recombinant host expressing the heterologous sequence. In some embodiments, a coding sequence is a sequence that is native to the host.

As used herein, the terms “variant” and “mutant” are used to describe a protein sequence that has been modified at one or more amino acids, compared to the wild-type sequence of a particular protein.

The invention described herein provides a method for producing commercial quantities of nootkatone to be used in flavorings, perfumes, and/or insecticides. The method employs a recombinant host capable of producing glycosylated nootkatol, which is nontoxic to the host, unlike the toxic compounds, nootkatol and nootkatone. The method involves detoxification of nootkatol via its glycosylation, which allows for the greater accumulation of non-toxic (now-glycosylated) nootkatol (a nootkatone precursor), which thereby ultimately allows for the greater production of nootkatone. Glycosylated nootkatol is rendered non-toxic by its glycosylation. Glycosylated nootkatol produced by the host can then be subsequently de-glycosylated and converted to nootkatone, as described below. Thus, biosynthesis of glycosylated nootkatol allows for production of larger quantities of nootkatone, as compared to methods of producing nootkatone that comprise a step of producing nootkatol in a host.

As used herein, the terms “modified nootkatol,” “nootkatol derivative,” and “nootkatol analog” can be used interchangeably to refer to a compound that can be derived from nootkatol or a compound with a similar structure to nootkatol.

As used herein, the terms “glycosylation,” “glycosylate,” “glycosylated,” and “protection group(s)” can be used interchangeably to refer to the chemical reaction in which a carbohydrate molecule is covalently attached to a hydroxyl group or attached to another functional group in a molecule capable of being covalently attached to a carbohydrate molecule. The term “mono” used in reference to glycosylation refers to the attachment of one carbohydrate molecule. The term “di” used in reference to glycosylation refers to the attachment of two carbohydrate molecules. The term “tri” used in reference to glycosylation refers to the attachment of three carbohydrate molecules. Additionally, the terms “oligo” and “poly” used in reference to a glycosylated molecule refers to the attachment of two or more carbohydrate molecules and can encompass molecules having a variety of attached carbohydrate molecules. As used herein, the terms “sugar,” “sugar moiety,” “sugar molecule,” “saccharide,” “saccharide moiety,” “saccharide molecule,” “carbohydrate,” “carbohydrate moiety,” and “carbohydrate molecule” can be used interchangeably.

As used herein, the terms “UDP-glycosyltransferase,” “glycosyltransferase,” and “UGT” are used interchangeably to refer to any enzyme capable of transferring sugar residues and derivatives thereof (including but not limited to galactose, xylose, rhamnose, glucose, arabinose, glucuronic acid, and others as understood in the art, e.g., N-acetyl glucosamine) to acceptor molecules. Acceptor molecules, such as, but not limited to, terpenes include, but are not limited to, other sugars, proteins, lipids, and other organic substrates, such as an alcohol and particularly nootkatol, as disclosed herein. The acceptor molecule can be termed an aglycon (aglucone if the sugar is glucose). An aglycon, includes, but is not limited to, the non-carbohydrate part of a glycoside. A “glycoside” as used herein refers an organic molecule with a glycosyl group (organic chemical group derived from a sugar or polysaccharide molecule) connected thereto by way of, for example, an intervening oxygen, nitrogen or sulphur atom. The product of glycosyl transfer can be an O-, N-, S-, or C-glycoside, and the glycoside can be a part of a monosaccharide, disaccharide, oligosaccharide, or polysaccharide. In particular aspects, the glycosyltransferase enzyme is a eukaryotic enzyme, i.e., an enzyme produced in a eukaryotic species including without limitation species from yeast, fungi, plants, and animals. In some embodiments, the glycosyltransferase enzyme is a bacterial enzyme.

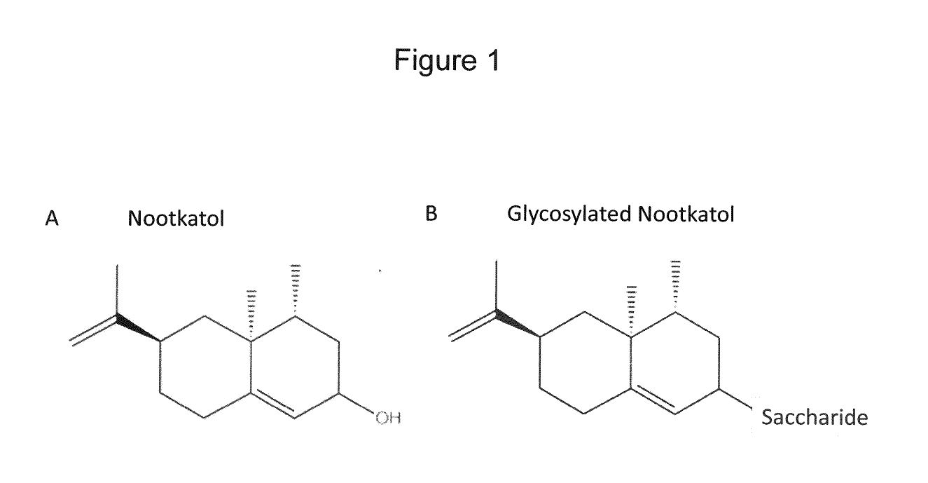

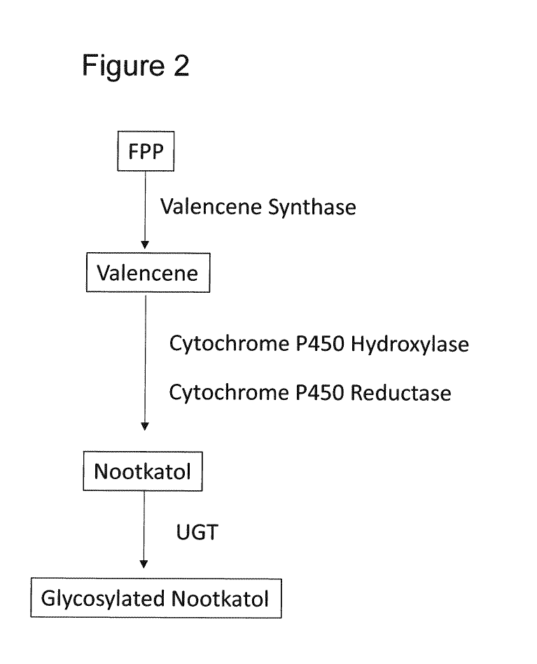

As used herein, the terms “nootkatol-glycoside” and “glycosylated nootkatol” can be used interchangeably to refer to nootkatol glycosylated at the hydroxyl group, wherein glycosylation comprises covalently attaching one or a plurality of saccharide moieties (FIG. 1). Glycosylated nootkatol and nootkatone precursors that are glycosylated can be produced in vivo (i.e., in a recombinant host), in vitro (i.e., enzymatically), or by whole cell bioconversion.

In some embodiments, glycosylated nootkatol and/or glycosylated nootkatol precursors are produced in vivo through expression of one or more enzymes involved in a glycosylated nootkatol biosynthetic pathway in a recombinant host. For example, a valencene-producing recombinant host expressing one or more of a gene encoding a cytochrome P450 polypeptide, a cytochrome P450 reductase polypeptide, and a UGT polypeptide can produce glycosylated nootkatol and glycosylated nootkatol precursors in vivo. In some embodiments, the cytochrome P450 polypeptide is a Hyoscyamus muticus cytochrome P450 hydroxylase (HPO; SEQ ID NO:1, SEQ ID NO:2) or a Cichorium intybus cytochrome P450 (SEQ ID NO:3, SEQ ID NO:4). In some embodiments, the cytochrome P450 reductase polypeptide is a Nicotiana sylvestris cytochrome P450 reductase polypeptide (SEQ ID NO:5, SEQ ID NO:6) or ATR1 (SEQ ID NO:7, SEQ ID NO:8). The UGT can be any UGT capable of glycosylating nootkatol. In some embodiments, the UGT is UGT85A1 (SEQ ID NO:9, SEQ ID NO:10), UGT76E1 (SEQ ID NO:11, SEQ ID NO:12), UGT73E1 (SEQ ID NO:13, SEQ ID NO:14), UGT73C3 (SEQ ID NO:15, SEQ ID NO:16), or UGT76E12 (SEQ ID NO:17, SEQ ID NO:18).

A valencene-producing host can be any host capable of producing valencene. Examples of valencene-producing recombinant hosts are described in U.S. Pat. No. 7,442,785, WO 2012/058636, and WO 2014/150599, each of which is incorporated by reference herein in its entirety. In some embodiments, a valencene-producing strain is S. cerevisiae strain, ALX11-30, comprising an Eryngium glaciale valencene synthase. See, e.g., WO 2012058636, WO 2014150599, and U.S. 2015/0007368, each of which has been incorporated by reference in its entirety. In some embodiments, the valence synthase is a valencene synthase encoded by a nucleotide sequence set forth in SEQ ID NO:19 and/or having an amino acid sequence set forth in SEQ ID NO:20. ALX11-30 was derived from S. cerevisiae strain, CALI5-1, which was derived from wild-type strain MATa, deposited under accession number ATCC 28383. See, e.g., U.S. Pat. No. 6,531,303, U.S. Pat. No. 6,689,593, and Takahashi et al., 2007, Biotechnol Bioeng. 97(1):170-81). CALI5-1 was generated to have decreased activity of the Dpp1 phosphatase (see, e.g., U.S. 20040249219). CALI5-1 comprises an ERG9 mutation (the Δerg9::HIS3 allele) as well as a mutation supporting aerobic sterol uptake enhancement. It also comprises approximately 8 copies of the truncated HMG2 gene. The truncated form of allows for an increase in carbon flow to FPP. It also contains a deletion in the gene encoding diacylglycerol pyrophosphate (DGPP) phosphatase enzyme (dpp1), which limits dephosphorylation of FPP. See, e.g., WO 2012058636, which has been incorporated by reference in its entirety.

In some embodiments, glycosylated nootkatol and/or glycosylated nootkatol precursors are produced through contact of a glycosylated nootkatol precursor with one or more enzymes involved in a glycosylated nootkatol pathway in vitro. For example, contacting nootkatol with a UGT polypeptide can result in production of glycosylated nootkatol in vitro. Non-limiting examples of UGTs capable of glycosylated nootkatol comprise UGT85A1 (SEQ ID NO:9, SEQ ID NO:10), UGT76E1 (SEQ ID NO:11, SEQ ID NO:12), UGT73E1 (SEQ ID NO:13, SEQ ID NO:14), UGT73C3 (SEQ ID NO:15, SEQ ID NO:16), or UGT76E12 (SEQ ID NO:17, SEQ ID NO:18).

In some embodiments, a glycosylated nootkatol precursor is produced through contact of an upstream glycosylated nootkatol precursor with one or more enzymes involved in a glycosylated nootkatol pathway in vitro. For example, contacting valencene with a cytochrome P450 and a cytochrome P450 reductase results in production of nootkatol.

In some embodiments, glycosylated nootkatol and/or glycosylated nootkatol precursors are produced by whole cell bioconversion. For whole cell bioconversion to occur, a host cell expressing one or more enzymes involved in a glycosylated nootkatol pathway takes up and modifies a glycosylated nootkatol precursor in the cell; following modification in vivo, glycosylated nootkatol can be excreted into the culture medium. In a non-limiting example, nootkatol is the glycosylated nootkatol precursor, and modification of the glycosylated nootkatol precursor refers to glycosylation of nootkatol. For example, a host cell expressing a gene encoding a UGT polypeptide can take up nootkatol and glycosylate nootkatol in the cell; following glycosylation in vivo, glycosylated nootkatol is excreted into the culture medium.

In some aspects, recombinant host cells are engineered such that at least one endogenous enzyme with activity capable of de-glycosylating glycosylated nootkatol is inhibited, down-regulated, functionally disrupted, or deleted. Such de-glycosylation activities include those capable of cleaving a saccharide from glycosylated nootkatol. In some embodiments, the at least one endogenous enzyme with activity capable of de-glycosylating glycosylated nootkatol that is preferably inhibited, down-regulated, disrupted or functionally deleted includes, but is not limited to, a β-glucosidase, a cellulase, a cellobiase, a β-galactosidase, and a β-glucuronidase. In some embodiments, the at least one endogenous host enzyme that is inhibited, down-regulated, disrupted or functionally deleted, is classified as EC number: 3.2.1.58. In some aspects, when the glycosylated nootkatol is produced in Saccharomyces cerevisiae, EXG1, may be inhibited, down-regulated, functionally disrupted, or deleted.

In some aspects, glycosylated nootkatol is less toxic to a host than nootkatol and/or nootkatone. In some aspects, glycosylated nootkatol is not toxic to a host. See Example 2. The non-toxic glycosylated nootkatol produced by a host can then be converted to nootkatol and subsequently to nootkatone to produce large quantities of nootkatone to be used in commercial applications.

In some aspects, glycosylated nootkatol produced in vivo, in vitro, or by bioconversion is subsequently isolated and/or purified. In some embodiments, glycosylated nootkatol is purified by flash chromatography or HPLC. In further aspects, the isolated and/or purified glycosylated nootkatol is further de-glycosylated to obtain nootkatol. In some embodiments, glycosylated nootkatol is de-glycosylated biocatalytically or chemically. Enzymes capable of cleaving a saccharide from glycosylated nootkatol include, but are not limited to, β-glucosidase, Depol™ (cellulase), cellulase T. reesei, glusulase, cellobiase A. niger, β-galactosidase A. oryzae, β-glucuronidase, and EXG1. Chemical methods for cleavage of a saccharide from glycosylated nootkatol include incubation of glycosylated nootkatol in acidic conditions. Non-limiting examples of acidic solutions include sulfuric acid, hydrochloric acid, camphor sulfonic acid, nitric acid, acetic acid, formic acid, trifluoroacetic acid, acetyl chloride, thionyl chloride, or other reagents capable of generating hydrochloric acid in situ. Additionally, a resin or polymer bearing acidic moieties can be used to cleave a saccharide from glycosylated nootkatol. The resins or polymer can be strongly acidic, typically featuring sulfonic acid moieties, such as Amberlite® (Sigma-Aldrich), or weakly acidic, typically featuring carboxylic acid groups or sulfonic acid moieties.

In still further aspects, de-glycosylated nootkatol is converted to nootkatone. Conversion of nootkatol to nootkatone can be performed either biocatalytically or chemically in vitro. Biocatalytic conversion of nootkatol to nootkatone can involve use of an alcohol dehydrogenase. Methods to chemically convert nootkatol to nootkatone can involve use of manganese dioxide, a chromic acid-derived reagent such as pyridinium chlorochromate (PCC) or pyridinium dichromate (PDC), aerobic oxidation catalyzed by copper, such as copper chloride, hydrogen transfer systems catalyzed by palladium such as palladium(II) acetate (Pd(OAc)₂) immobilized on a support, such as a charcoal support, 2,3-Dichloro-5,6-Dicyanobenzoqunone (DDQ) peroxides such as tert-butyl hydroperoxide or hydrogen peroxide (H₂O₂), meta-Chloroperoxybenzoic acid (mCPBA), hypervalent iodine reagents, silver carbonate, ruthenium reagents such as tetrapropylammonium perruthenase, periodates, zirconium reagents, methods based on DMSO reduction, such as Swern oxidation or related, sulfur trioxide-based methods, or Oppenauer oxidation methods.

As used herein, the term “detectable concentration” refers to a level of valencene, glycosylated nootkatol, nootkatol, and/or nootkatone measured in units including, but not limited to, AUC, OD₆₀₀, mg/L, μg/L, μM, or mM. Valencene, glycosylated nootkatol, nootkatol, and/or nootkatone production can be detected and/or analyzed by techniques generally available to one skilled in the art, for example, but not limited to, thin layer chromatography (TLC), high-performance liquid chromatography (HPLC), ultraviolet-visible spectroscopy/spectrophotometry (UV-Vis), mass spectrometry (MS), and nuclear magnetic resonance spectroscopy (NMR). In some aspects, glycosylated nootkatol is produced at concentrations of approximately 10 mg/L.

As used herein, the terms “or” and “and/or” is utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” In some embodiments, “and/or” is used to refer to the exogenous nucleic acids that a recombinant cell comprises, wherein a recombinant cell comprises one or more exogenous nucleic acids selected from a group. In some embodiments, “and/or” is used to refer to production of glycosylated nootkatol and/or glycosylated nootkatol precursors. In some embodiments, “and/or” is used to refer to production of glycosylated nootkatol, wherein one or more glycosylated nootkatol molecules are produced. In some embodiments, “and/or” is used to refer to production of glycosylated nootkatol, wherein one or more glycosylated nootkatol molecules are produced through one or more of the following steps: culturing a recombinant microorganism, synthesizing one or more glycosylated nootkatol molecules in a recombinant microorganism, and/or isolating one or more glycosylated nootkatol molecules.

### Functional Homologs

Functional homologs of the polypeptides described above are also suitable for use in producing glycosylated nootkatol in a recombinant host. A functional homolog is a polypeptide that has sequence similarity to a reference polypeptide, and that carries out one or more of the biochemical or physiological function(s) of the reference polypeptide. A functional homolog and the reference polypeptide can be a natural occurring polypeptide, and the sequence similarity can be due to convergent or divergent evolutionary events. As such, functional homologs are sometimes designated in the literature as homologs, orthologs, or paralogs. Variants of a naturally occurring functional homolog, such as polypeptides encoded by mutants of a wild type coding sequence, can themselves be functional homologs. Functional homologs can also be created via site-directed mutagenesis of the coding sequence for a polypeptide, or by combining domains from the coding sequences for different naturally occurring polypeptides (“domain swapping”). Techniques for modifying genes encoding functional polypeptides described herein are known and include, inter alia, directed evolution techniques, site-directed mutagenesis techniques and random mutagenesis techniques, and can be useful to increase specific activity of a polypeptide, alter substrate specificity, alter expression levels, alter subcellular location, or modify polypeptide-polypeptide interactions in a desired manner. Such modified polypeptides are considered functional homologs. The term “functional homolog” is sometimes applied to the nucleic acid that encodes a functionally homologous polypeptide.

Functional homologs can be identified by analysis of nucleotide and polypeptide sequence alignments. For example, performing a query on a database of nucleotide or polypeptide sequences can identify homologs of glycosylated nootkatol biosynthesis polypeptides. Sequence analysis can involve BLAST, Reciprocal BLAST, or PSI-BLAST analysis of non-redundant databases using a UGT amino acid sequence as the reference sequence. Amino acid sequence is, in some instances, deduced from the nucleotide sequence. Those polypeptides in the database that have greater than 40% sequence identity are candidates for further evaluation for suitability as a glycosylated nootkatol biosynthesis polypeptide. Amino acid sequence similarity allows for conservative amino acid substitutions, such as substitution of one hydrophobic residue for another or substitution of one polar residue for another. If desired, manual inspection of such candidates can be carried out in order to narrow the number of candidates to be further evaluated. Manual inspection can be performed by selecting those candidates that appear to have domains present in glycosylated nootkatol biosynthesis polypeptides, e.g., conserved functional domains.

Conserved regions can be identified by locating a region within the primary amino acid sequence of a glycosylated nootkatol biosynthesis polypeptide that is a repeated sequence, forms some secondary structure (e.g., helices and beta sheets), establishes positively or negatively charged domains, or represents a protein motif or domain. See, e.g., the Pfam web site describing consensus sequences for a variety of protein motifs and domains on the World Wide Web at sanger.ac.uk/Software/Pfam/ and pfam.janelia.org/. The information included at the Pfam database is described in Sonnhammer et al., Nucl. Acids Res., 26:320-322 (1998); Sonnhammer et al., Proteins, 28:405-420 (1997); and Bateman et al., Nucl. Acids Res., 27:260-262 (1999). Conserved regions also can be determined by aligning sequences of the same or related polypeptides from closely related species. Closely related species preferably are from the same family. In some embodiments, alignment of sequences from two different species is adequate to identify such homologs.

Typically, polypeptides that exhibit at least about 40% amino acid sequence identity are useful to identify conserved regions. Conserved regions of related polypeptides exhibit at least 45% amino acid sequence identity (e.g., at least 50%, at least 60%, at least 70%, at least 80%, or at least 90% amino acid sequence identity). In some embodiments, a conserved region exhibits at least 92%, 94%, 96%, 98%, or 99% amino acid sequence identity.

For example, polypeptides suitable for producing glycosylated nootkatol in a recombinant host include functional homologs of UGTs.

Methods to modify the substrate specificity of, for example, a UGT, are known to those skilled in the art, and include without limitation site-directed/rational mutagenesis approaches, random directed evolution approaches and combinations in which random mutagenesis/saturation techniques are performed near the active site of the enzyme. For example, see Osmani et al., 2009, Phytochemistry 70: 325-347.

A candidate sequence typically has a length that is from 80% to 200% of the length of the reference sequence, e.g., 82, 85, 87, 89, 90, 93, 95, 97, 99, 100, 105, 110, 115, 120, 130, 140, 150, 160, 170, 180, 190, or 200% of the length of the reference sequence. A functional homolog polypeptide typically has a length that is from 95% to 105% of the length of the reference sequence, e.g., 90, 93, 95, 97, 99, 100, 105, 110, 115, or 120% of the length of the reference sequence, or any range between. A percent identity for any candidate nucleic acid or polypeptide relative to a reference nucleic acid or polypeptide can be determined as follows. A reference sequence (e.g., a nucleic acid sequence or an amino acid sequence described herein) is aligned to one or more candidate sequences using the computer program ClustalW (version 1.83, default parameters), which allows alignments of nucleic acid or polypeptide sequences to be carried out across their entire length (global alignment). Chenna et al., 2003, Nucleic Acids Res. 31(13):3497-500.

ClustalW calculates the best match between a reference and one or more candidate sequences, and aligns them so that identities, similarities and differences can be determined. Gaps of one or more residues can be inserted into a reference sequence, a candidate sequence, or both, to maximize sequence alignments. For fast pairwise alignment of nucleic acid sequences, the following default parameters are used: word size: 2; window size: 4; scoring method: % age; number of top diagonals: 4; and gap penalty: 5. For multiple sequence alignments of nucleic acid sequences, the following parameters are used: gap opening penalty: 10.0; gap extension penalty: 5.0; and weight transitions: yes. For fast pairwise alignment of protein sequences, the following parameters are used: word size: 1; window size: 5; scoring method: % age; number of top diagonals: 5; gap penalty: 3. For multiple alignment of protein sequences, the following parameters are used: weight matrix: blosum; gap opening penalty: 10.0; gap extension penalty: 0.05; hydrophilic gaps: on; hydrophilic residues: Gly, Pro, Ser, Asn, Asp, Gln, Glu, Arg, and Lys; residue-specific gap penalties: on. The ClustalW output is a sequence alignment that reflects the relationship between sequences. ClustalW can be run, for example, at the Baylor College of Medicine Search Launcher site on the World Wide Web (searchlauncher.bcm.tmc.edu/multi-align/multi-align.html) and at the European Bioinformatics Institute site on the World Wide Web (ebi.ac.uk/clustalw).

To determine a % identity of a candidate nucleic acid or amino acid sequence to a reference sequence, the sequences are aligned using ClustalW, the number of identical matches in the alignment is divided by the length of the reference sequence, and the result is multiplied by 100. It is noted that the % identity value can be rounded to the nearest tenth. For example, 78.11, 78.12, 78.13, and 78.14 are rounded down to 78.1, while 78.15, 78.16, 78.17, 78.18, and 78.19 are rounded up to 78.2.

It will be appreciated that functional UGT proteins can include additional amino acids that are not involved in the enzymatic activities carried out by the enzymes. In some embodiments, UGT proteins are fusion proteins. The terms “fusion protein” and “chimeric protein” can be used interchangeably refer to proteins engineered through the joining of two or more genes that code for different proteins. In some embodiments, a nucleic acid sequence encoding a UGT polypeptide can include a tag sequence that encodes a “tag” designed to facilitate subsequent manipulation (e.g., to facilitate purification or detection), secretion, or localization of the encoded polypeptide. Tag sequences can be inserted in the nucleic acid sequence encoding the UGT polypeptide such that the encoded tag is located at either the carboxyl or amino terminus of the UGT polypeptide. Non-limiting examples of encoded tags include green fluorescent protein (GFP), glutathione S transferase (GST), HIS tag, and Flag™ tag (Kodak, New Haven, Conn.). Other examples of tags include a chloroplast transit peptide, a mitochondrial transit peptide, an amyloplast peptide, signal peptide, or a secretion tag.

### Glycosylated Nootkatol Biosynthesis Nucleic Acids

A recombinant gene encoding a polypeptide described herein comprises the coding sequence for that polypeptide, operably linked in sense orientation to one or more regulatory regions suitable for expressing the polypeptide. Because many microorganisms are capable of expressing multiple gene products from a polycistronic mRNA, multiple polypeptides can be expressed under the control of a single regulatory region for those microorganisms, if desired. A coding sequence and a regulatory region are considered to be operably-linked when the regulatory region and coding sequence are positioned so that the regulatory region is effective for regulating transcription or translation of the sequence. Typically, the translation initiation site of the translational reading frame of the coding sequence is positioned between one and about fifty nucleotides downstream of the regulatory region for a monocistronic gene.

In many cases, the coding sequence for a polypeptide described herein is identified in a species other than the recombinant host, i.e., is a heterologous nucleic acid. Thus, if the recombinant host is a microorganism, the coding sequence can be from other prokaryotic or eukaryotic microorganisms, from plants or from animals. In some case, however, the coding sequence is a sequence that is native to the host and is being reintroduced into that organism. A native sequence can often be distinguished from the naturally occurring sequence by the presence of non-natural sequences linked to the exogenous nucleic acid, e.g., non-native regulatory sequences flanking a native sequence in a recombinant nucleic acid construct. In addition, stably transformed exogenous nucleic acids typically are integrated at positions other than the position where the native sequence is found. “Regulatory region” refers to a nucleic acid having nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, introns, and combinations thereof. A regulatory region typically comprises at least a core (basal) promoter. A regulatory region also can include at least one control element, such as an enhancer sequence, an upstream element or an upstream activation region (UAR). A regulatory region is operably linked to a coding sequence by positioning the regulatory region and the coding sequence so that the regulatory region is effective for regulating transcription or translation of the sequence. For example, to operably link a coding sequence and a promoter sequence, the translation initiation site of the translational reading frame of the coding sequence is typically positioned between one and about fifty nucleotides downstream of the promoter. A regulatory region can, however, be positioned as much as about 5,000 nucleotides upstream of the translation initiation site, or about 2,000 nucleotides upstream of the transcription start site.

The choice of regulatory regions to be included depends upon several factors, including, but not limited to, efficiency, selectability, inducibility, desired expression level, and preferential expression during certain culture stages. It is a routine matter for one of skill in the art to modulate the expression of a coding sequence by appropriately selecting and positioning regulatory regions relative to the coding sequence. It will be understood that more than one regulatory region can be present, e.g., introns, enhancers, upstream activation regions, transcription terminators, and inducible elements.

### Recombinant Hosts

Recombinant hosts can be used to express polypeptides for producing glycosylated nootkatol, including mammalian, insect, plant, and algal cells. A number of prokaryotes and eukaryotes are also suitable for use in constructing the recombinant microorganisms described herein, e.g., gram-negative bacteria, yeast, and fungi. A strain selected for use as a glycosylated nootkatol production strain is first analyzed to determine which production genes are endogenous to the strain and which genes are not present. Genes for which an endogenous counterpart is not present in the strain are advantageously assembled in one or more recombinant constructs, which are then transformed into the strain in order to supply the missing function(s).

The constructed and genetically engineered microorganisms provided by the invention can be cultivated using conventional fermentation processes, including, inter alia, chemostat, batch, fed-batch cultivations, continuous perfusion fermentation, and continuous perfusion cell culture.

Carbon sources of use in the instant method include any molecule that can be metabolized by the recombinant host cell to facilitate growth and/or production of glycosylated nootkatol, Examples of suitable carbon sources include, but are not limited to, sucrose (e.g., as found in molasses), fructose, xylose, ethanol, glycerol, glucose, cellulose, starch, cellobiose or other glucose comprising polymer. In embodiments employing yeast as a host, for example, carbons sources such as sucrose, fructose, xylose, ethanol, glycerol, and glucose are suitable. The carbon source can be provided to the host organism throughout the cultivation period or alternatively, the organism can be grown for a period of time in the presence of another energy source, e.g., protein, and then provided with a source of carbon only during the fed-batch phase.

Exemplary prokaryotic and eukaryotic species are described in more detail below. However, it will be appreciated that other species can be suitable. For example, suitable species can be in a genus such as Agaricus, Aspergillus, Bacillus, Candida, Corynebacterium, Eremothecium, Escherichia, Fusarium/Gibberella, Kluyveromyces, Laetiporus, Lentinus, Phaffia, Phanerochaete, Pichia, Physcomitrella, Rhodoturula, Saccharomyces, Schizosaccharomyces, Sphaceloma, Xanthophyllomyces or Yarrowia. Exemplary species from such genera include Lentinus tigrinus, Laetiporus sulphureus, Phanerochaete chrysosporium, Pichia pastoris, Cyberlindnera jadinii, Physcomitrella patens, Rhodoturula glutinis 32, Rhodoturula mucilaginosa, Phaffia rhodozyma U BV-AX, Xanthophyllomyces dendrorhous, Fusarium fujikuroi/Gibberella fujikuroi, Candida utilis, Candida glabrata, Candida albicans, and Yarrowia lipolytica.

In some embodiments, a microorganism can be a prokaryote such as Escherichia coli, Rhodobacter sphaeroides, Rhodobacter capsulatus, or Rhodotorula toruloides or a eukaryote such as Saccharomyces cerevisiae.

In some embodiments, a microorganism can be an Ascomycete such as Gibberella fujikuroi, Kluyveromyces lactis, Schizosaccharomyces pombe, Aspergillus niger, Yarrowia lipolytica, Ashbya gossypii, or Saccharomyces cerevisiae.

In some embodiments, a microorganism can be an algal cell such as Blakeslea trispora, Dunaliella salina, Haematococcus pluvialis, Chlorella sp., Undaria pinnatifida, Sargassum, Laminaria japonica, or Scenedesmus almeriensis species.

In some embodiments, a microorganism can be a cyanobacterial cell such as Blakeslea trispora, Dunaliella salina, Haematococcus pluvialis, Chlorella sp., Undaria pinnatifida, Sargassum, Laminaria japonica, or Scenedesmus almeriensis.

### Saccharomyces spp.

Saccharomyces is a widely used chassis organism in synthetic biology, and can be used as the recombinant microorganism platform. For example, there are libraries of mutants, plasmids, detailed computer models of metabolism and other information available for S. cerevisiae, allowing for rational design of various modules to enhance product yield. Methods are known for making recombinant microorganisms.

### Aspergillus spp.

Aspergillus species such as A. oryzae, A. niger and A. sojae are widely used microorganisms in food production and can also be used as the recombinant microorganism platform. Nucleotide sequences are available for genomes of A. nidulans, A. fumigatus, A. oryzae, A. clavatus, A. flavus, A. niger, and A. terreus, allowing rational design and modification of endogenous pathways to enhance flux and increase product yield. Metabolic models have been developed for Aspergillus. Generally, A. niger is cultured for the industrial production of a number of food ingredients such as citric acid and gluconic acid, and thus species such as A. niger are generally suitable for producing glycosylated nootkatol.

Escherichia coli

Escherichia coli, another widely used platform organism in synthetic biology, can also be used as the recombinant microorganism platform. Similar to Saccharomyces, there are libraries of mutants, plasmids, detailed computer models of metabolism and other information available for E. coli, allowing for rational design of various modules to enhance product yield. Methods similar to those described above for Saccharomyces can be used to make recombinant E. coli microorganisms.

Agaricus, Gibberella, and Phanerochaete spp.

Agaricus, Gibberella, and Phanerochaete spp. can be useful because they are known to produce large amounts of isoprenoids in culture. Thus, the terpene precursors for producing large amounts of glycosylated nootkatol are already produced by endogenous genes. Thus, modules comprising recombinant genes for glycosylated nootkatol biosynthesis polypeptides can be introduced into species from such genera without the necessity of introducing mevalonate or MEP pathway genes.

Arxula adeninivorans (Blastobotrys adeninivorans)

Arxula adeninivorans is a dimorphic yeast (it grows as a budding yeast like the baker's yeast up to a temperature of 42° C., above this threshold it grows in a filamentous form) with unusual biochemical characteristics. It can grow on a wide range of substrates and can assimilate nitrate. It has successfully been applied to the generation of strains that can produce natural plastics or the development of a biosensor for estrogens in environmental samples.

Yarrowia lipolytica.

Yarrowia lipolytica is a dimorphic yeast (see Arxula adeninivorans) and belongs to the family Hemiascomycetes. The entire genome of Yarrowia lipolytica is known. Yarrowia species is aerobic and considered to be non-pathogenic. Yarrowia is efficient in using hydrophobic substrates (e.g. alkanes, fatty acids, oils) and can grow on sugars. It has a high potential for industrial applications and is an oleaginous microorganism. Yarrowia lipolyptica can accumulate lipid content to approximately 40% of its dry cell weight and is a model organism for lipid accumulation and remobilization. See e.g., Nicaud, 2012, Yeast 29(10):409-18; Beopoulos et al., 2009, Biohimie 91(6):692-6; Bankar et al., 2009, Appl Microbiol Biotechnol. 84(5):847-65.

### Rhodotorula sp.

Rhodotorula is a unicellular, pigmented yeast. The oleaginous red yeast, Rhodotorula glutinis, has been shown to produce lipids and carotenoids from crude glycerol (Saenge et al., 2011, Process Biochemistry 46(1):210-8). Rhodotorula toruloides strains have been shown to be an efficient fed-batch fermentation system for improved biomass and lipid productivity (Li et al., 2007, Enzyme and Microbial Technology 41:312-7).

Rhodosporidium toruloides

Rhodosporidium toruloides is an oleaginous yeast and useful for engineering lipid-production pathways (See e.g. Zhu et al., 2013, Nature Commun. 3:1112; Ageitos et al., 2011, Applied Microbiology and Biotechnology 90(4):1219-27).

Candida boidinii

Candida boidinii is methylotrophic yeast (it can grow on methanol). Like other methylotrophic species such as Hansenula polymorpha and Pichia pastoris, it provides an excellent platform for producing heterologous proteins. Yields in a multigram range of a secreted foreign protein have been reported. A computational method, IPRO, recently predicted mutations that experimentally switched the cofactor specificity of Candida boidinii xylose reductase from NADPH to NADH. See, e.g., Mattanovich et al., 2012, Methods Mol Biol. 824:329-58; Khoury et al., 2009, Protein Sci. 18(10):2125-38.

Hansenula polymorpha (Pichia angusta)

Hansenula polymorpha is methylotrophic yeast (see Candida boidinii). It can furthermore grow on a wide range of other substrates; it is thermo-tolerant and can assimilate nitrate (see also Kluyveromyces lactis). It has been applied to producing hepatitis B vaccines, insulin and interferon alpha-2a for the treatment of hepatitis C, furthermore to a range of technical enzymes. See, e.g., Xu et al., 2014, Virol Sin. 29(6):403-9.

Kluyveromyces lactis

Kluyveromyces lactis is yeast regularly applied to the production of kefir. It can grow on several sugars, most importantly on lactose, which is present in milk and whey. It has successfully been applied among others for producing chymosin (an enzyme that is usually present in the stomach of calves) for producing cheese. Production takes place in fermenters on a 40,000 L scale. See, e.g., van Ooyen et al., 2006, FEMS Yeast Res. 6(3):381-92.

Pichia pastoris

Pichia pastoris is methylotrophic yeast (see Candida boidinii and Hansenula polymorpha). It provides an efficient platform for producing foreign proteins. Platform elements are available as a kit and it is worldwide used in academia for producing proteins. Strains have been engineered that can produce complex human N-glycan (yeast glycans are similar but not identical to those found in humans). See, e.g., Piirainen et al., 2014, N Biotechnol. 31(6):532-7.

### Physcomitrella spp.

Physcomitrella mosses, when grown in suspension culture, have characteristics similar to yeast or other fungal cultures. This genera can be used for producing plant secondary metabolites, which can be difficult to produce in other types of cells.

### Methods of Producing Glycosylated Nootkatol

Recombinant hosts described herein comprising optimized UGT genes can be used in methods to produce glycosylated nootkatol. For example, the method can include growing the recombinant host in a culture medium under conditions in which glycosylated nootkatol biosynthesis genes are expressed. The recombinant host can be grown in a fed batch or continuous process. Typically, the recombinant host is grown in a fermentor at a defined temperature(s) for a desired period of time. Depending on the particular host used in the method, other recombinant genes such as isopentenyl biosynthesis genes and terpene synthase and cyclase genes can also be present and expressed. Levels of substrates and intermediates, e.g., valencene and nootkatol, can be determined by extracting samples from culture media for analysis according to published methods.

After the recombinant host has been grown in culture for the desired period of time, glycosylated nootkatol can then be recovered (i.e., isolated) from the culture using various techniques known in the art. In some embodiments, a permeabilizing agent can be added to aid the feedstock entering into the host and product getting out. For example, a crude lysate of the cultured microorganism can be centrifuged to obtain a supernatant. The resulting supernatant can then be applied to a chromatography column, e.g., a C-18 column, and washed with water to remove hydrophilic compounds, followed by elution of the compound(s) of interest with a solvent such as methanol. The compound(s) can then be further purified by preparative HPLC.

It will be appreciated that the various genes and modules discussed herein can be present in two or more recombinant hosts rather than a single host. When a plurality of recombinant host is used, they can be grown in a mixed culture to produce glycosylated nootkatol.

Alternatively, the two or more hosts each can be grown in a separate culture medium and the product of the first culture medium, e.g., nootkatol, can be introduced into second culture medium to be converted into a subsequent intermediate, or into an end product such as, for example, glycosylated nootkatol. The product produced by the second, or final host is then recovered. It will also be appreciated that in some embodiments, a recombinant host is grown using nutrient sources other than a culture medium and utilizing a system other than a fermentor.

The invention will be further described in the following examples, which do not limit the scope of the invention described in the claims.

### EXAMPLES

The Examples that follow are illustrative of specific embodiments of the invention, and various uses thereof. They, are set forth for explanatory purposes only, and are not to be taken as limiting the invention.

### Example 1. In Vitro Glycosylation of Nootkatol

UGT85A1 (SEQ ID NO:9, SEQ ID NO:10), UGT76E1 (SEQ ID NO:11, SEQ ID NO:12), UGT73E1 (SEQ ID NO:13, SEQ ID NO:14), UGT73C3 (SEQ ID NO:15, SEQ ID NO:16), and UGT76E12 (SEQ ID NO:17, SEQ ID NO:18) were each cloned into a T7 promoter-based vector comprising a sequence coding for an N-terminal 6×His-tag. The vector backbone was linearized with restriction endonucleases, the UGT genes were amplified by PCR, and the constructs were verified by DNA sequencing.

Competent E. coli expression cells were transformed individually with a UGT-comprising plasmid. A colony from each transformation was inoculated individually in 6 mL NZCYM broth comprising an antibiotic. The pre-culture was incubated overnight at 37° C. and 220 rpm and used to inoculate 100 mL NZYCM broth with an antibiotic at an initial OD₆₀₀ of 0.2. After growth at 37° C. until an OD₆₀₀ of 0.6-0.8, the cells were induced for protein expression using 0.2 mM IPTG, followed by incubation at 20° C. and 120 rpm for 18-20 h.

Cells were harvested by centrifugation at approximately 4° C. and approximately 4000 g for 20 min and resuspended in 3 mL 10 mM Tris-HCl, pH 8.0 plus protease inhibitor. After cell disruption, 6 mL 1×HIS binding buffer (20 mM Tris-HCl, pH 7.5, 500 mM NaCl, and 10 mM imidazole) were added to the suspension. Cell crude extracts were centrifuged at 4° C. and approximately 4000 g for 30 min. The supernatant was collected, and 300 μL of nickel beads were added; the mixture was incubated with gentle mixing at 4° C. for 2 h. The mixture was centrifuged at 4° C. and approximately 1000 g for 3 min, the supernatant was removed, and the beads were resuspended with 3 mL 1×HIS binding buffer. This step was performed twice. The beads were then resuspended and mixed gently in 500 μL 1×HIS Binding Buffer. The mixture was then transferred into a cold Eppendorf tube, centrifuged at 4° C. and approximately 1000 g, and the supernatant was removed. The beads were resuspended in 400 μL elution buffer (20 mM Tris-HCl, pH 7.5, 500 mM NaCl, 250 mM imidazole), mixed gently, centrifuged at 4° C. and approximately 1000 g for 3 min, and the supernatant was collected. This step was performed three times to collect three elution fractions. Glycerol was added in a 1:1 ratio to each elution tube and protein solutions were stored at −80° C. The fractions were analyzed by SDS-PAGE and Western Blot. The fraction comprising the UGT with the highest purity was used for the subsequent in vitro assay.

UGT85A1 (SEQ ID NO:9, SEQ ID NO:10), UGT76E1 (SEQ ID NO:11, SEQ ID NO:12), UGT73E1 (SEQ ID NO:13, SEQ ID NO:14), UGT73C3 (SEQ ID NO:15, SEQ ID NO:16), and UGT76E12 (SEQ ID NO:17, SEQ ID NO:18) were employed for in vitro glycosylation of nootkatol in the presence of the sugar donor, UDP-glucose. The reaction was carried out in a final volume of 50 μL buffer (100 mM Tris, pH 8.0, 5 mM MgCl₂, 1 mM KCl), 5 mM nootkatol in DMSO, 15 mM UDP-glucose, and 5 μL purified enzyme solution. The reaction was incubated overnight at 30° C. The sample was analyzed by LC-MS on a BEH C18-column (100 mm×2.1 mm, 5 μm). Mobile phase A was 0.1% formic acid in water; mobile phase B was 0.1% formic acid in acetonitrile. B concentration gradient was 0-1 min, 1%; 1-5 min, 100%; 5-7 min, 100%; 7-7.1 min, 1%; 7.1-9 min, 1%. The injection volume was 5 μL. Mass spectrometry analysis was carried out on an SQD1 detector (3.4 KV capillary, 37V cone, 3 V extractor, 0.1 V RF lens) at a source temperature of 150° C. and desolvation temperature of 250° C., with full scan ESI+/−(100-900 amu scan range) and selective ion recording. The areas of the peaks corresponding to glycosylated nootkatol (nootkatol+1 glucose) are shown in Table 1.

TABLE 1
Area-under-peak values for glycosylated
nootkatol produced in vitro.

| | | Area- |
|---|---|---|
| | | under- |
| UGT | Organism | curve |
| UGT85A1 | Arabidopsis thaliana | 1632282 |
| (SEQ ID NO: 9, SEQ ID NO: 10) | | |
| UGT76E1 | Arabidopsis thaliana | 1566336 |
| (SEQ ID NO: 11, SEQ ID NO: 12) | | |
| UGT73E1 | Stevia rebaudiana | 1288384 |
| (SEQ ID NO: 13, SEQ ID NO: 14) | | |
| UGT73C3 | Arabidopsis thaliana | 1211635 |
| (SEQ ID NO: 15, SEQ ID NO: 16) | | |
| UGT76E12 | Arabidopsis thaliana | 820053 |
| (SEQ ID NO: 17, SEQ ID NO: 18) | | |

### Example 2. Analysis of the Growth-Inhibitory Effect of Nootkatol and Glycosylated Nootkatol on Yeast

A 20 mL seed culture of wild-type MATa strain ATCC 28383 was grown in SD-THUL medium (0.67 Bacto yeast nitrogen base without amino acids, 2% glucose, 0.14% yeast synthetic drop-out medium without uracil, tryptophan, histidine and leucine). The culture was grown for 24 h, and 2.5 mL of the culture was used to inoculate 7 equal batches of 50 mL fermentation medium (2% (NH₄)₂SO₄, 2% KH₂PO₄, 0.1% NaCl, 0.6% MgSO₄.7H₂O, 0.4% yeast extract, 1 mL mineral solution [FeSO₄.7H₂O 0.028%, ZnSO₄.7H2O 0.029%, CuSO₄.5H2O 0.008%, Na₂MoO₄.2H₂O 0.024%, CoCl₂.6H₂O 0.024%, MnSO₄.H₂O 0.017%, HCl 1 mL], 0.5 mL 50% glucose, 1.5 mL vitamin solution [biotin 0.001%, Ca-pantothenate 0.012%, inositol 0.06%, pyridoxine-HCl 0.012%, thiamine-HCl 0.012%], and 0.5 mL 10% CaCl₂) in 250 mL baffled Ehrlenmeyer flasks.

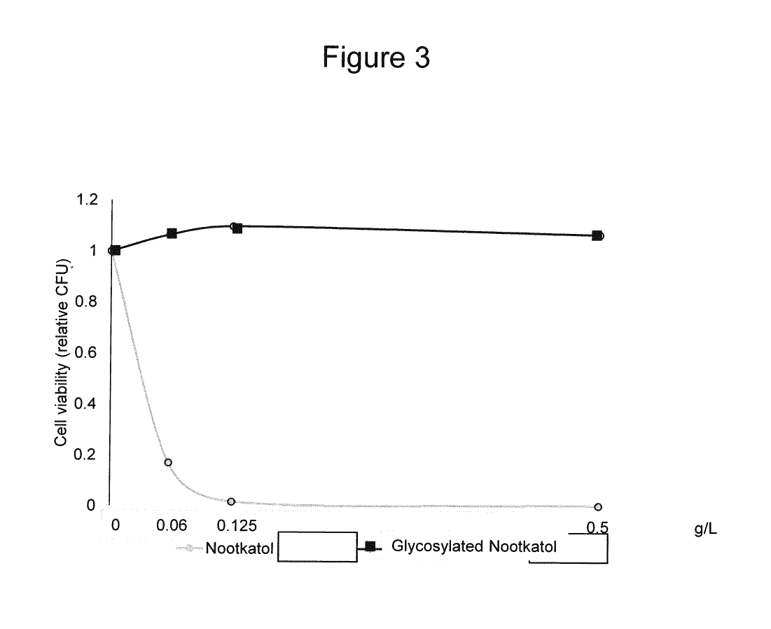

In 6 flasks, nootkatol or glycosylated nootkatol (nootkatol+1 glucose) were added in final concentrations of 0.06, 0.125, and 0.5 g/L. The 7^{th} flask was used as a control and was not treated with nootkatol or glycosylated nootkatol. The cultures were grown at 28° C. and 170 rpm, and the cell viability was measured after 5 h of exposure by plating 100 μL of 1/1000 dilution of cell culture on yeast extract peptone dextrose (YPD) plates. As shown in FIG. 3, nearly 99% cell death occurred upon addition of 0.125 g/L nootkatol, but no toxicity was observed even at 0.5 g/L glycosylated nootkatol.

### Example 3. Construction of S. cerevisiae Strain Producing Glycosylated Nootkatol

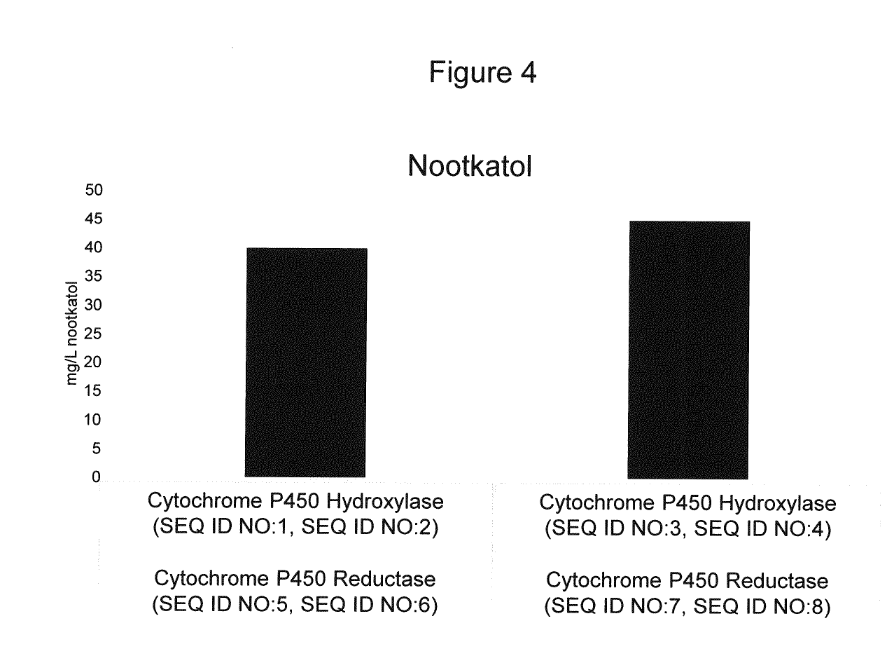

An expression vector comprising a Hyoscyamus muticus cytochrome P450 hydroxylase (SEQ ID NO:1, SEQ ID NO:2) and a Nicotiana cytochrome P450 reductase (SEQ ID NO:5, SEQ ID NO:6) or an expression vector comprising a Chicorium intybus cytochrome p450 hydroxylase (SEQ ID NO:3, SEQ ID NO:4) and ATR1 (SEQ ID NO:7, SEQ ID NO:8) was transformed into a valencene-producing Saccharomyces cerevisiae strain further comprising Eryngium glaciale valence synthase (SEQ ID NO:19, SEQ ID NO:20). Eight colonies were analyzed for nootkatol production in a shake flask. 20 mL seed cultures were started in SD-THUL medium in 250 mL flasks using freshly growing colonies and grown for 24 h. 2.0 mL of the starter culture was used to inoculate 50 mL of fermentation medium+2% soybean oil in a 250 mL baffled flask. The cultures were grown for 16 h at 28° C. and 170 rpm. The cultures were then fed 2 mL of 50% glucose and 0.39 mL of 12.5% yeast extract. The pH of the culture was adjusted to 4.5 using NaOH 6 h after feeding. After another 18 h, the cultures were fed 3 mL 50% glucose and 0.63 mL of 12.5% yeast extract, and the pH was once again adjusted to 4.5 with NaOH 6 h later. After 18 h, the cultures were fed for the third time 4 mL 50% glucose and 0.89 mL of 12.5% yeast extract, and the pH of the cultures was adjusted to 4.5 using NaOH 6 h later. The following day, a 2 mL culture sample was extracted with 2 mL acetone and subsequently extracted with 4 mL of a hexane/hexadecane solution. An aliquot was analyzed by GC to determine nootkatol levels, which are shown in FIG. 4.

UGT85A1 (SEQ ID NO:9, SEQ ID NO:10), UGT76E1 (SEQ ID NO:11, SEQ ID NO:12), or UGT73E1 (SEQ ID NO:13, SEQ ID NO:14) were cloned into the plasmid comprising a cytochrome p450 hydroxylase and a cytochrome p450 reductase, as discussed above. The valencene-producing strain was then individually transformed with a plasmid. Yeast strains were freshly streaked on SD-THUL plates. 20 mL seed cultures were inoculated from the freshly grown plates in 250 mL flasks in SD-THUL medium. The culture was grown for 24 h, and 2.0 mL of this culture was used to inoculate 50 mL fermentation medium with or without 2% soybean oil in a 250 mL baffled flask. The cultures were grown as described above. After 4 days, a sample was analyzed for valencene, nootkatol, and glycosylated nootkatol levels. For levels secreted in the medium with or without oil, the supernatant of the growth culture sample was extracted with ethyl acetate. To analyze the total production of valencene, nootkatol, and glycosylated nootkatol, 2 mL whole culture were directly extracted with 2 mL acetone and then extracted with 4 mL hexane/hexadecane solution.

Nootkatol was identified by GC-MS by its dehydrated component nootkatene (M⁺ 202) and comparing retention time and mass spectra against those of authentic standard of beta-nootkatol and comparing against MS library spectra in Wiley Library FFNSC 2.0—Flavors and Fragrances of Natural and Synthetic Compounds—Mass Spectral Database. GC-MS was conducted using a Perkin Elmer TurboMass GC-MS with electron impact (EI) ionization, fitted with a ZB-5MSi (Phenomenex, 5% Phenyl 95% Dimethylpolysiloxane Phase, 30 m×0.25 mm×0.25 μm) non-polar GC capillary column. The following conditions were used: injector temperature 250° C., ion source temperature 280° C., GC-interface line temperature 250° C., oven temperature program 50° C., hold for 2 min, 8° C./min to 100° C., hold 0 min and 18° C./min to 225° C., hold 4 min, run time 19.2 min, solvent delay 5 min, carrier gas-He at 1 mL/min, injection 1 μL, split 1:10, scan range of 40-500 m/z acquiring in 0.2 s at 70 eV.

Glycosylated nootkatol formation was measured by LC-MS-MS. Samples were prepared by diluting whole culture samples 1:1 with ethyl acetate in a 2 mL tube comprising 0.5 g acid-washed glass beads. Samples were disrupted by orbital agitation at 6,500 rpm for 3×20 second pulses at ˜4° C. Disrupted cell samples were clarified by centrifugation at 12,000 rpm and 4° C. for 2 min. Extracted organic phase (upper layer) was then transferred to a new 2 mL tube and dried under vacuum at 55° C. for ˜15 min or until all traces of organic solvent were removed. Dried extracts were reconstituted in 0.5 mL of 100% methanol and, if necessary, filtered using a 0.22 μm nylon spin filter at 8,000 rpm for 1 min at ambient temperature.

Twenty microliters of each sample was separated using a Kinetex EVO C18 column (5 μm; 4.6×50 mm) from Phenomenex with an isocratic non-aqueous reversed phase (NARP) isocratic LC program consisting of 80% A (100% methanol with 0.1% formic acid) and 20% B (100% isopropanol with 0.1% formic acid). The duration of the LC program was 8 min, and the LC system (including mobile phase and column) were held isothermic at 30° C. Samples were ionized using the Turbo V source as a front end to the API3200 triple quadrupole MS (AB SciEx) operating in atmospheric pressure chemical ionization (APCI) mode. N₂ was used as both the collision and source gases. Source parameters were as follows: current 35V, 400° C., source/gas 1 (GS1, 40), source/gas 2 (GS 2, 40), interface heater status on, dissociation gas flow (CAD, 3), nebulizing current+/−3. Analytes were detected and quantified in MRM mode with rapid toggle between positive and negative ionization modes (Table 2). Data acquisition, instrument command, and data analysis were all performed using Analyst 1.6.2 software.

TABLE 2
Data acquisition, instrument command, and data analysis parameters.

| | 1^{st} | 2^{nd} | | | | | | | |
|---|---|---|---|---|---|---|---|---|---|
| | Quadrupole | Quadrupole | | | | Collision | | Collision | |
| | Mass | Mass | | Declustering | Exit | Entrance | Collision | Exit | |
| | Filter | Filter | | Potential | Potential | Potential | Energy | Potential | |
| | (Q1) | (Q3) | Time | (DP) | (EP) | (CEP) | (CE) | (CXP) | |
| | (Dalton) | (Dalton) | (ms) | (Volts) | (Volts) | (Volts) | (Volts) | (Volts) | Mode |
| Nootkatol | 217 | 111 | 150 | 31.58 | 6.47 | 13.20 | 23.38 | 2.83 | Pos |
| Glycosylated | 381 | 100 | 150 | −49.91 | −8.73 | −21.98 | −22.00 | −4.47 | Neg |
| nootkatol | | | | | | | | | |

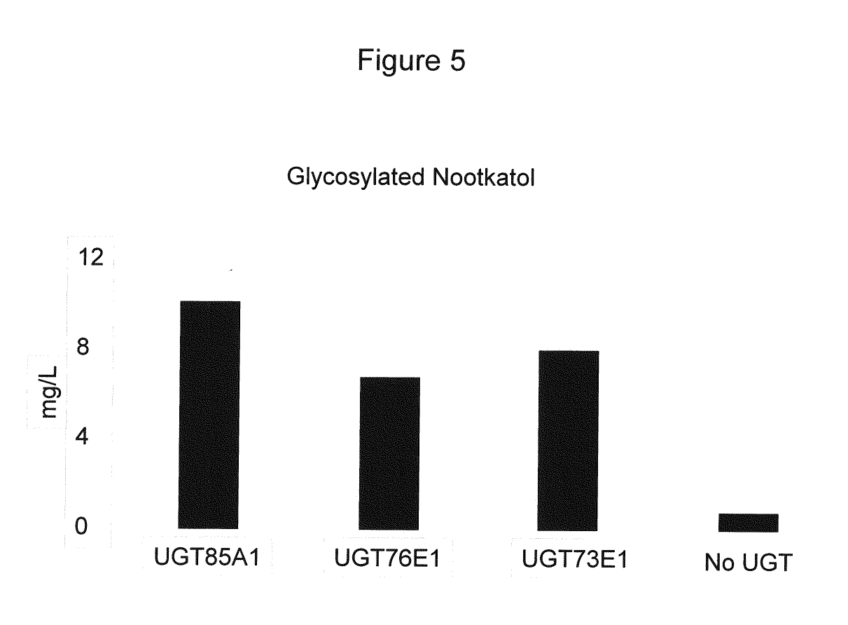

As shown in FIG. 5, nootkatol-producing S. cerevisiae strains comprising Eryngium glaciale valence synthase (SEQ ID NO:19, SEQ ID NO:20), Chicorium intybus cytochrome p450 hydroxylase (SEQ ID NO:3, SEQ ID NO:4), Arabidopsis thaliana cytochrome p450 reductase (SEQ ID NO:7, SEQ ID NO:6), and either UGT85A1 (SEQ ID NO:9, SEQ ID NO:10), UGT76E1 (SEQ ID NO:11, SEQ ID NO:12), or UGT73E1 (SEQ ID NO:13, SEQ ID NO:14) produced approximately 10 mg/L glycosylated nootkatol.

### Example 4. De-Glycosylation of Glycosylated Nootkatol to Generate Nootkatol

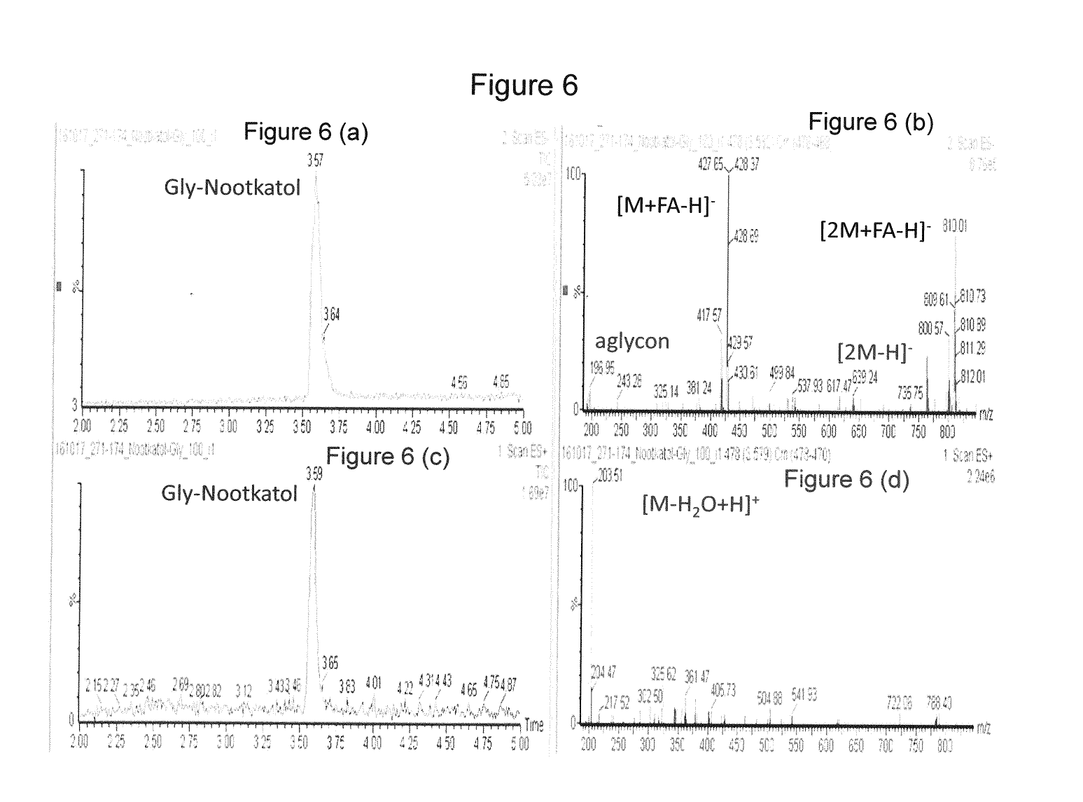

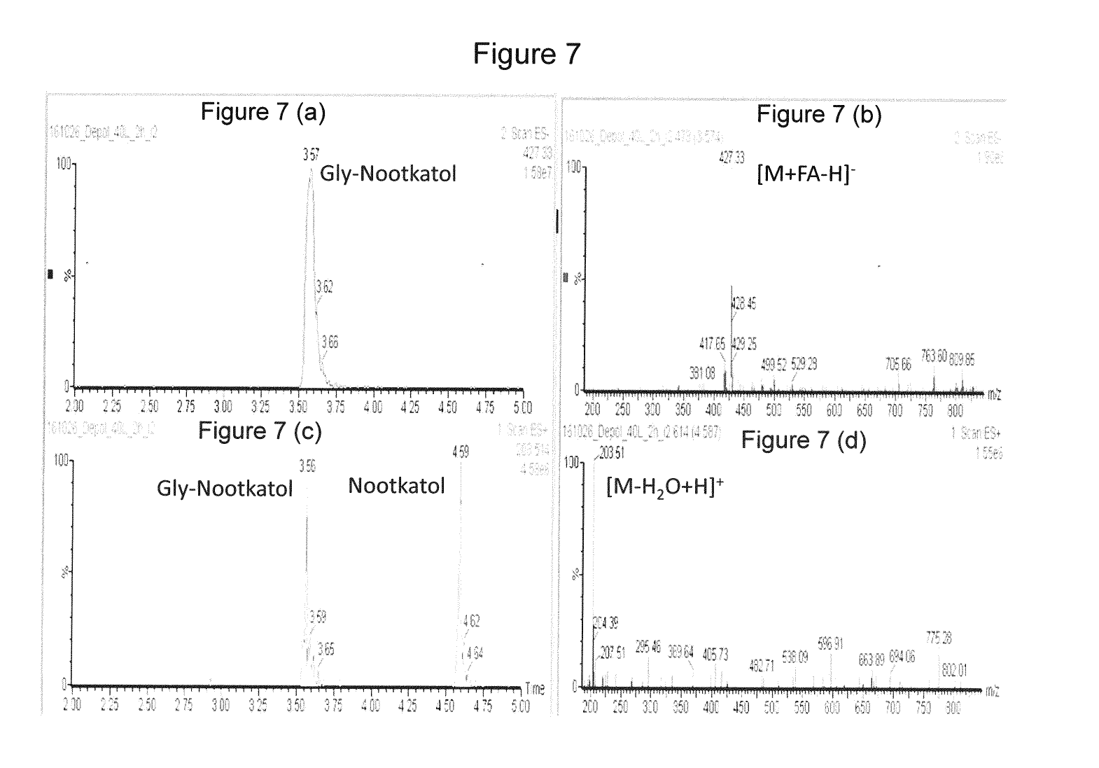

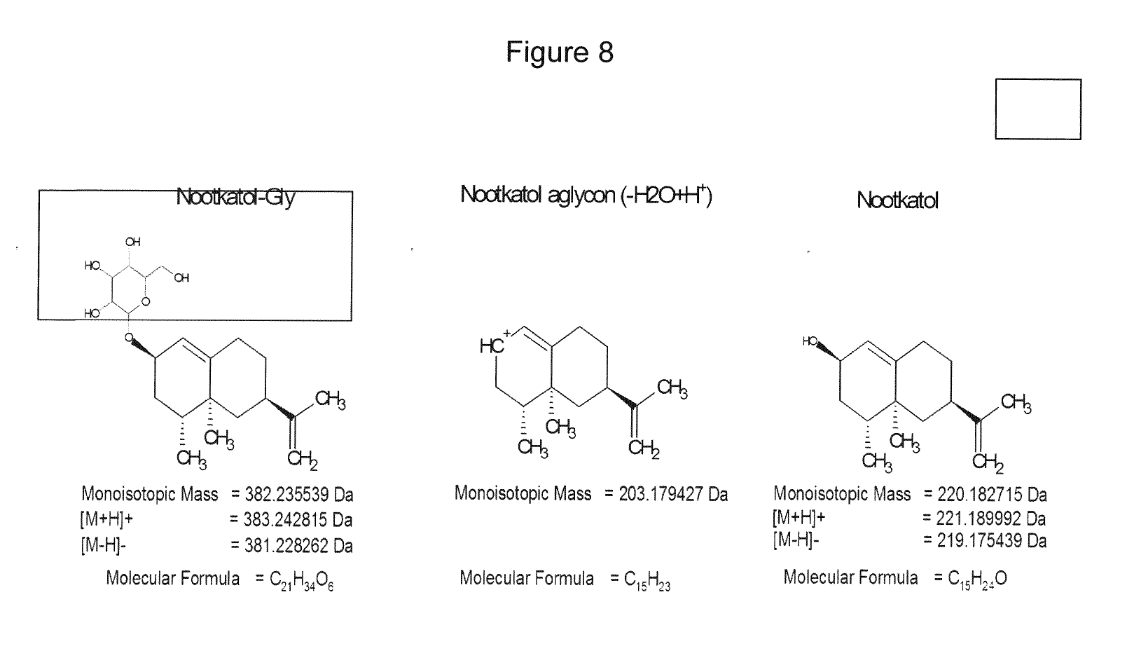

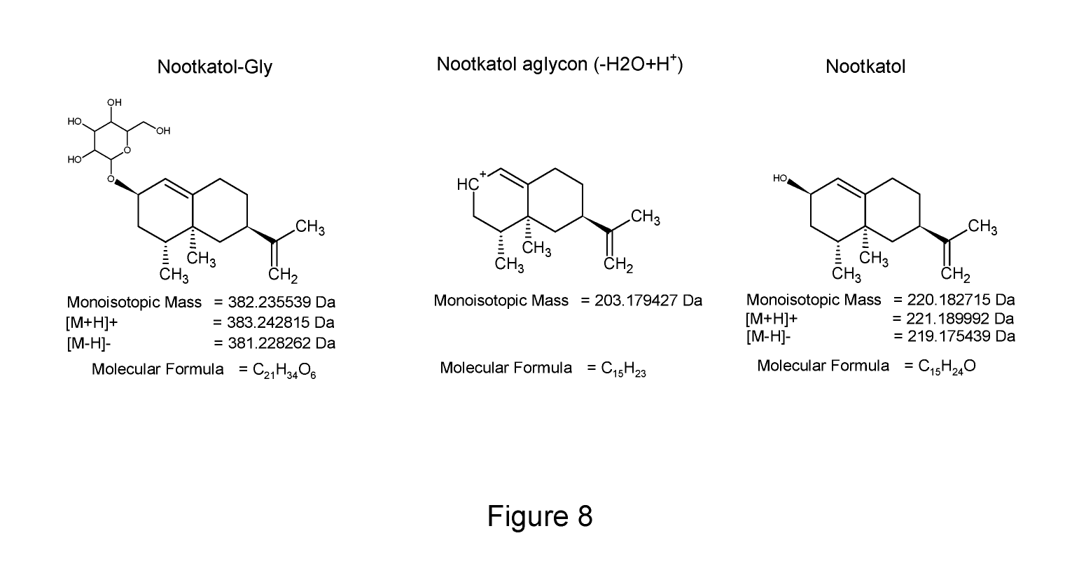

In vitro cleavage of sugar moieties of glycosylated nootkatol and subsequent isolation of nootkatol from culture medium was performed (see FIGS. 6-8) as follows.

Confirmation of reaction substrates was performed using NMR experiments in DMSO-d6 at 25° C. using a Bruker Avance III 400 MHz NMR spectrometer equipped with a 5 mm CPPBBO BB-1H/19F/D Z-GRD probe. The structure was solved by means of one-dimensional standard homo-nuclear multipulse NMR experiments.

Identical samples of 990 μl of Delft fermentation medium further comprising 0.1 mg nootkatol-glucoside (Nootkatol-Glc) were each treated with 10 μl of 12 exemplary glycosidase enzymes (listed in Table 3 below) (thus, 1% v/v) for 2 hours. After 2 hours, samples of the reaction mixture were taken, and the de-glycosylation reaction was terminated by addition of an equal volume of ethanol followed by freezing.

The resulting digested samples were analyzed by LC-MS on a BEH C18-column (100 mm×2.1 mm, 5 μm). Mobile phase A was 0.1% formic acid in water, and mobile phase B was 0.1% formic acid in acetonitrile. B concentration gradient was 0-1 min, 1%; 1-5 min, 100%; 5-7 min, 100%; 7-7.1 min, 1%; 7.1-9 min, 1%. The injection volume was 5 μL. Mass spectrometry analysis was carried out on an SQD1 detector (3.4 KV capillary, 37 V cone, 3 V extractor, 0.1 V RF lens) at a source temperature of 150° C. and desolvation temperature of 250° C., with full scan ESI+/−(100-900 amu scan range) and selective ion recording. The areas of the peaks corresponding to de-glycosylated nootkatol-Glc (nootkatol) are shown in Table 3. Table 3 shows production of nootkatol from nootkatol-Glc with varying efficiency for all of the commercially available glycosidases tested, as well as a weak spontaneous de-glycosylation in the water control during the course of the conditions.

TABLE 3
Area under curve (AUC) integrating each peak of the
LC-MS using the selected ion chromatogram of m/z 203.5.
The values thereby indicate the relative efficiency
of de-glycosylation of each enzyme tested.

| Sample | Enzyme | Gly-Nootkatol | Nootkatol |
|---|---|---|---|
| 1 | Depol_40L | 418435.72 | 376235.63 |
| 2 | Depol_670L | 459452.16 | 358522.78 |
| 3 | Depol_692L | 1031493.69 | 317604.13 |
| 4 | G016L | 273656.88 | 589872.38 |
| 5 | CX15L | 64761.65 | 1056271.13 |
| 6 | TSE2017 | 321005.09 | 1027914.38 |
| 7 | NS11033 | 425473.5 | 497991.38 |
| 8 | NS11034 | 453300.41 | 549358.88 |
| 9 | NS11035 | 487782.72 | 386400.5 |
| 10 | NS11036 | 674636.13 | 40644.02 |
| 11 | NS11037 | 525042.88 | 31963.28 |
| 12 | NS11038 | 627254.19 | 32917.85 |
| 13 | H₂O | 682080.13 | 8803.77 |

### Example 5. Reduction of Background De-Glycosylation Activity in Host to Elevate Yield of Glycosylated Nootkatol

Exg1 is an exo-1,3-beta-glucanases (EC number: 3.2.1.58) endogenous to S. cerevisiae (see Table 4). It is hypothesized that it can deglycosylate the UGT-mediated glycosylated-nootkatol in vivo. Therefore, it is anticipated that deletion or disruption of at least one endogenous exo-1,3-beta-glucanase increases the production of glycosylated-nootkatol by the yeast. These enzymes are preferred targets for disruption in glycosylated nootkatol producing S. cerevisiae recombinant hosts.

TABLE 4
Exg1, an endogenous S. cerevisiae enzyme
capable of in vivo deglycosylation of
glycosylated nootkatol.

| Name | Sequence |
|---|---|
| S288C_ | ATGCTTTCGCTTAAAACGTTACTGTGTACGTTGTTGACTG |
| YLR300W_ | TGTCATCAGTACTCGCTACCCCAGTCCCTGCAAGAGACCC |
| EXG1 | TTCTTCCATTCAATTTGTTCATGAGGAGAACAAGAAAAGA |
| (SEQ ID | TACTACGATTATGACCACGGTTCCCTCGGAGAACCAATCC |
| NO: 21) | GTGGTGTCAACATTGGTGGTTGGTTACTTCTTGAACCATA |
| | CATTACTCCATCTTTGTTCGAGGCTTTCCGTACAAATGAT |
| | GACAACGACGAAGGAATTCCTGTCGACGAATATCACTTCT |
| | GTCAATATTTAGGTAAGGATTTGGCTAAAAGCCGTTTACA |
| | GAGCCATTGGTCTACTTTCTACCAAGAACAAGATTTCGCT |
| | AATATTGCTTCCCAAGGTTTCAACCTTGTCAGAATTCCTA |
| | TCGGTTACTGGGCTTTCCAAACTTTGGACGATGATCCTTA |
| | TGTTAGCGGCCTACAGGAATCTTACCTAGACCAAGCCATC |
| | GGTTGGGCTAGAAACAACAGCTTGAAAGTTTGGGTTGATT |
| | TGCATGGTGCCGCTGGTTCGCAGAACGGGTTTGATAACTC |
| | TGGTTTGAGAGATTCATACAAGTTTTTGGAAGACAGCAAT |
| | TTGGCCGTTACTACAAATGTCTTGAACTACATATTGAAAA |
| | AATACTCTGCGGAGGAATACTTGGACACTGTTATTGGTAT |
| | CGAATTGATTAATGAGCCATTGGGTCCTGTTCTAGACATG |
| | GATAAAATGAAGAATGACTACTTGGCACCTGCTTACGAAT |
| | ACTTGAGAAACAACATCAAGAGTGACCAAGTTATCATCAT |
| | CCATGACGCTTTCCAACCATACAATTATTGGGATGACTTC |
| | ATGACTGAAAACGATGGCTACTGGGGTGTCACTATCGACC |
| | ATCATCACTACCAAGTCTTTGCTTCTGATCAATTGGAAAG |
| | ATCCATTGATGAACATATTAAAGTAGCTTGTGAATGGGGT |
| | ACCGGAGTTTTGAATGAATCCCACTGGACTGTTTGTGGTG |
| | AGTTTGCTGCCGCTTTGACTGATTGTACAAAATGGTTGAA |
| | TAGTGTTGGCTTCGGCGCTAGATACGACGGTTCTTGGGTC |
| | AATGGTGACCAAACATCTTCTTACATTGGCTCTTGTGCTA |
| | ACAACGATGATATAGCTTACTGGTCTGACGAAAGAAAGGA |
| | AAACACAAGACGTTATGTGGAGGCACAACTAGATGCCTTT |
| | GAAATGAGAGGGGGTTGGATTATCTGGTGTTACAAGACAG |
| | AATCTAGTTTGGAATGGGATGCTCAAAGATTGATGTTCAA |
| | TGGTTTATTCCCTCAACCATTGACTGACAGAAAGTATCCA |
| | AACCAATGTGGCACAATTTCTAACTAA |
| Amino | MLSLKTLLCTLLTVSSVLATPVPARDPSSIQFVHEENKKR |
| acid | YYDYDHGSLGEPIRGVNIGGWLLLEPYITPSLFEAFRTND |
| sequence | DNDEGIPVDEYHFCQYLGKDLAKSRLQSHWSTFYQEQDFA |
| of | NIASQGFNLVRIPIGYWAFQTLDDDPYVSGLQESYLDQAI |
| S288C_ | GWARNNSLKVWVDLHGAAGSQNGFDNSGLRDSYKFLEDSN |
| YLR300W_ | LAVTTNVLNYILKKYSAEEYLDTVIGIELINEPLGPVLDM |
| EXG1 | DKMKNDYLAPAYEYLRNNIKSDQVIIIHDAFQPYNYWDDF |
| (SEQ ID | MTENDGYWGVTIDHHHYQVFASDQLERSIDEHIKVACEWG |
| NO: 22) | TGVLNESHWTVCGEFAAALTDCTKWLNSVGFGARYDGSWV |
| | NGDQTSSYIGSCANNDDIAYWSDERKENTRRYVEAQLDAF |
| | EMRGGWIIWCYKTESSLEWDAQRLMFNGLFPQPLTDRKYP |
| | NQCGTISN |

Having described the invention in detail and by reference to specific embodiments thereof, it will be apparent that modifications and variations are possible without departing from the scope of the invention defined in the appended claims. More specifically, although some aspects of the present invention are identified herein as particularly advantageous, it is contemplated that the present invention is not necessarily limited to these particular aspects of the invention.

What is claimed is:

1. A recombinant host comprising:
(a) a gene encoding a valencene synthase polypeptide;
(b) a gene encoding a cytochrome P450 hydroxylase polypeptide;
(c) a gene encoding a cytochrome P450 reductase polypeptide; and/or
(d) a gene encoding a glycosyltransferase (UGT) polypeptide, wherein the UGT polypeptide is capable of glycosylating nootkatol,
wherein at least one of said genes is a recombinant gene, and wherein the recombinant host produces glycosylated nootkatol.

2. The recombinant host of claim 1, wherein
(a) the valencene synthase polypeptide comprises a valencene synthase polypeptide having at least 50% identity to an amino acid sequence set forth in SEQ ID NO:20;
(b) the cytochrome P450 hydroxylase polypeptide comprises a cytochrome P450 hydroxylase polypeptide having at least 50% identity to an amino acid sequence set forth in SEQ ID NO:2 or SEQ ID NO:4;
(c) the cytochrome P450 reductase polypeptide comprises a cytochrome P450 reductase polypeptide having at least 50% identity to an amino acid sequence set forth in SEQ ID NO:6 or SEQ ID NO:8; and/or
(d) the UGT polypeptide comprises a UGT polypeptide having at least 50% identity to an amino acid sequence set forth in SEQ ID NO:10, SEQ ID NO:12, SEQ ID NO:14, SEQ ID NO:16, OR SEQ ID NO:18.

3. The recombinant host of claim 1 or claim 2, wherein the glycosylated nootkatol comprises monoglycosylated, diglycosylated, triglycosylated, or polyglycosylated nootkatol.

4. The recombinant host of any one of claims 1-3, wherein the recombinant host is characterized by a relative colony-forming unit (CFU) value of at least 0.9.

5. The recombinant host of any one of claims 1-4, wherein the glycosylated nootkatol produced is not toxic to the recombinant host.

6. The recombinant host of any one of claims 1-5, wherein the host further comprises a downregulated, deleted or functionally disrupted endogenous gene encoding an enzyme capable of cleaving a saccharide from glycosylated nootkatol.

7. A method of producing glycosylated nootkatol, comprising:
(a) growing the recombinant host of any one of claims 1-6 in a culture medium;
wherein the glycosylated nootkatol is synthesized by the recombinant host; and
(b) optionally isolating the glycosylated nootkatol.

8. A method for producing glycosylated nootkatol from a bioconversion reaction, comprising:
(a) growing a recombinant host in a culture medium;
wherein the host comprises a gene encoding a UGT polypeptide capable of in vivo glycosylation of nootkatol and optionally functionally disrupting an endogenous gene encoding an enzyme capable of cleaving a saccharide from glycosylated nootkatol;
wherein the gene encoding the UGT polypeptide is expressed in the recombinant host;
(b) contacting the recombinant host with nootkatol in a reaction buffer to produce glycosylated nootkatol; and
(c) optionally isolating the glycosylated nootkatol.

9. The method of claim 8, wherein the UGT polypeptide comprises a UGT polypeptide having at least 50% identity to an amino acid sequence set forth in SEQ ID NO:10, SEQ ID NO:12, SEQ ID NO:14, SEQ ID NO:16, OR SEQ ID NO:18.

10. The method of any one of claims 7-9, further comprising a step of cleavage of sugar moieties of the glycosylated nootkatol, wherein nootkatol can be isolated from the culture medium.

11. The method of claim 10, wherein the step of cleavage of the sugar moieties of the glycosylated nootkatol comprises enzymatic cleavage.

12. The method of claim 11, wherein enzymatic cleavage comprises treating the culture medium with an enzyme capable of cleaving sugar moieties.

13. The method of claim 12, wherein the enzyme comprises β-glucosidase, cellulase, cellobiase, β-galactosidase, β-glucuronidase, or EXG1.

14. The method of claim 10, wherein the step of cleavage of the sugar moieties of the glycosylated nootkatol comprises chemical cleavage.

15. The method of claim 14, wherein chemical cleavage comprises treating the culture medium with a weak acid or under other conditions capable of cleaving sugar moieties.

16. The method of claim 15, wherein the weak acid comprises an organic acid or an inorganic acid.

17. The recombinant host recited in any one of claims 1-16, wherein the recombinant host comprises a microorganism that is a plant cell, a mammalian cell, an insect cell, a fungal cell, or a bacterial cell.

18. The recombinant host of claim 17, wherein the bacterial cell comprises Escherichia bacteria cells, Lactobacillus bacteria cells, Lactococcus bacteria cells, Cornebacterium bacteria cells, Acetobacter bacteria cells, Acinetobacter bacteria cells, or Pseudomonas bacterial cells.

19. The recombinant host of claim 17, wherein the fungal cell is a yeast cell.

20. The recombinant host of claim 19, wherein the yeast cell is a cell from Saccharomyces cerevisiae, Schizosaccharomyces pombe, Yarrowia lipolytica, Candida glabrata, Ashbya gossypii, Cyberlindnera jadinii, Pichia pastoris, Kluyveromyces lactis, Hansenula polymorpha, Candida boidinii, Arxula adeninivorans, Xanthophyllomyces dendrorhous, or Candida albicans species.

21. The recombinant host of claim 20, wherein the yeast cell is a Saccharomycete.

22. The recombinant host of claim 21, wherein the yeast cell is a cell from the Saccharomyces cerevisiae species.

23. The recombinant host cell of claim 22, wherein the yeast cell comprises a downregulated, deleted or functionally disrupted EXG1.

24. A method for producing glycosylated nootkatol from an in vitro reaction comprising contacting nootkatol with one or more UGT polypeptides in the presence of one or more UDP-sugars.

25. The method of claim 24, wherein the UGT polypeptide comprises a UGT polypeptide having at least 50% identity to an amino acid sequence set forth in SEQ ID NO:10, SEQ ID NO:12, SEQ ID NO:14, SEQ ID NO:16, OR SEQ ID NO:18.

26. The method of claim 24 or claim 25, wherein the one or more UDP-sugars comprise UDP-glucose, UDP-rhamnose, or UDP-xylose.

27. The method of any one of claim 8-16 or 24-26, wherein the nootkatol comprises plant-derived or synthetic nootkatol.

28. The method of any one of claim 8-16 or 24-27, further comprising a step of converting nootkatol to nootkatone.

29. The method of claim 28, wherein the step of converting nootkatol to nootkatone comprises chemical or biocatalytic conversion of nootkatol to nootkatone.

30. The method of any one of claim 7-16 or 24-29, further comprising a step of detecting the isolated glycosylated nootkatol, nootkatol, and/or nootkatone by thin layer chromatography (TLC), high-performance liquid chromatography (HPLC), ultraviolet-visible spectroscopy/spectrophotometry (UV-Vis), liquid chromatography-mass spectrometry (LC-MS), or nuclear magnetic resonance (NMR).

31. A glycosylated nootkatol composition produced by the recombinant host of any one of claim 1-6 or 24-27 or the method of any one of claim 7-9 or 24-27.

32. A nootkatol composition produced by the method of any one of claims 10-16.

33. A nootkatone composition produced by the method of claim 28 or claim 29.

34. The nootkatone composition of claim 33, wherein the nootkatone composition is used in a flavoring, a perfume, and/or an insect repellent.


SEQUENCE LISTING (22 sequences)

SEQ ID NO: 1 - DNA, 1509 bp, Hyoscyamus muticus
atgcaattcttcagcttggtttccatcttcctttttctatcttttttgtttttgttaaggaaatggaagaactccaatagccagtccaagaaattgcctccaggtccatggaaacttccattactaggtagcatgcttcatatggttggtggacttccacatcatgtacttagagatttagcaaaaaaatatggaccacttatgcatcttcaacttggtgaagtttctgctgttgttgttacttctcctgatatggcaaaagaagtactaaaaactcatgacattgcgttcgcgtctaggcctaaacttttagccccagagattgtatgttacaacaggtctgacattgcgttttgcccttatggtgattactggagacaaatgcgtaaaatttgtgtcttggaagtgttgagtgccaagaatgttaggtcattcagctctattaggcgcgatgaagtgcttcgtctagttaattttgtccgatcatctacgagtgagccggttaactttactgaaaggctgtttttattcacaagttccatgacatgtagatcagcatttgggaaagtgttcaaggaacaggaaacatttatacaactaatcaaagaagtgataggtttagcaggaggatttgatgtggctgacatcttcccatcactgaagttcctccatgtactaactggaatggagggtaagattatgaaggctcaccataaagtagatgcaattgttgaggatgtcatcaatgagcataagaagaaccttgcaatggggaaaactaatggtgcattaggaggtgaagatctaattgatgttcttttaagacttatgaatgatggaggccttcaatttcctatcaccaatgacaacatcaaagctattatctttgacatgtttgctgctgggacagagacttcatcgtcaacacttgtatgggctatggtgcaaatgatgagaaatccaactatactagccaaagctcaagcagaagtaagagaagcattcaaaggaaaagaaactttcgatgaaaatgatgtcgaagagttgaaatacttgaagttagtcattaaagaaactctaagactccatccaccagttccacttttggtcccaagagaatgtagggaagaaacagaaataaatggctacactattccagtaaagaccaaagtcatggttaatgtttgggcattaggaagagatccgaaatactgggacgacgcagataacttcaagccagagagatttgagcagtgttctgtggactttataggtaacaattttgaatatcttccatttggtggtggaaggaggatatgtccagggatatcatttggtttagctaatgtttatttgccattggctcaattgctatatcattttgattggaaactccctactggaatggaaccaaaagacttggatttgacagaattggttggaataactattgccagaaagagtgatcttatgttggttgcgactccttatcaaccttctcgagagtaa

SEQ ID NO: 2 - protein, 502 aa, Hyoscyamus muticus
MQFFSLVSIFLFLSFLFLLRKWKNSNSQSKKLPPGPWKLPLLGSMLHMVGGLPHHVLRDLAKKYGPLMHLQLGEVSAVVVTSPDMAKEVLKTHDIAFASRPKLLAPEIVCYNRSDIAFCPYGDYWRQMRKICVLEVLSAKNVRSFSSIRRDEVLRLVNFVRSSTSEPVNFTERLFLFTSSMTCRSAFGKVFKEQETFIQLIKEVIGLAGGFDVADIFPSLKFLHVLTGMEGKIMKAHHKVDAIVEDVINEHKKNLAMGKTNGALGGEDLIDVLLRLMNDGGLQFPITNDNIKAIIFDMFAAGTETSSSTLVWAMVQMMRNPTILAKAQAEVREAFKGKETFDENDVEELKYLKLVIKETLRLHPPVPLLVPRECREETEINGYTIPVKTKVMVNVWALGRDPKYWDDADNFKPERFEQCSVDFIGNNFEYLPFGGGRRICPGISFGLANVYLPLAQLLYHFDWKLPTGMEPKDLDLTELVGITIARKSDLMLVATPYQPSRE

SEQ ID NO: 3 - DNA, 1490 bp, Chicorium intybus
atggagatttctatccccactacccttggccttgccgtcatcatcttcatcattttcaagttgctaacgcgtaccacatcaaagaaaaacctactcccagagccatggagactaccaataatcggacacatgcatcatctgataggtacgatgccacatcgtggtgtcatggaactagccaggaagcatggatctctcatgcatctacaacttggagaagtgtccactattgtggtctcatccccacgttgggcaaaagaggttctgacaacgtacgatattacgtttgcaaacagaccggagactttaaccggtgagattgttgcatatcacaataccgatattgtccttgctccgtatggtgaatactggaggcagttgcgaaaactttgcaccttggagcttttaagcaacaagaaagtgaagtcgtttcagtcccttcgtgaggaggaatgttggaatctggttaaagacattcgatcaactgggcagggatccccaatcaatctttcagaaaacattttcaagatgattgccaccatacttagtagggcagcattcggaaagggaatcaaagaccaaatgaaatttacagaattagtaaaagaaatactaaggcttacgggaggttttgatgtggcggacatctttccttctaaaaagttacttcaccatctttcaggcaagagagctaagttaaccaacatacacaataaacttgacaatttgatcaacaatatcatcgctgagcaccctggaaaccgtacaagctcatcacaggagactctacttgatgttctgttaagactgaaagaaagcgcagagtttccattgacagcagacaatgtcaaagcagtcattttggatatgtttggagctggcacggatacttcgtcagccacaattgaatgggcaatctcagaattgataaggtgtccgagagccatggagaaagttcaaacagaattaaggcaagcactaaatggaaaggaaaggatccaagaagaagatctacaggaactaaattacctaaagctagtgatcaaagaaacattgaggttgcatccaccactaccgttggttatgcctagagagtgtagggagccatgtgtgttggggggatacgatatacccagcaagacgaaacttattgtcaacgtgtttgccataaacagggatcctgaatactggaaagatgctgaaactttcatgccagagagatttgaaaacagccccatcactgtaatgggttcagagtatgagtatctcccgtttggtgcaggaagaagaatgtgtccaggcgctgcccttggtttagccaacgtggaacttcctcttgctcatatactttactacttcaattggaagctcccaaatggaaaaacatttgaagacttggacatgactgagagctttggagccactgtccaaagaaagacggagttgttactagttccaacggatttccaaacacttacggcatctactta

SEQ ID NO: 4 - protein, 496 aa, Chicorium intybus
MEISIPTTLGLAVIIFIIFKLLTRTTSKKNLLPEPWRLPIIGHMHHLIGTMPHRGVMELARKHGSLMHLQLGEVSTIVVSSPRWAKEVLTTYDITFANRPETLTGEIVAYHNTDIVLAPYGEYWRQLRKLCTLELLSNKKVKSFQSLREEECWNLVKDIRSTGQGSPINLSENIFKMIATILSRAAFGKGIKDQMKFTELVKEILRLTGGFDVADIFPSKKLLHHLSGKRAKLTNIHNKLDNLINNIIAEHPGNRTSSSQETLLDVLLRLKESAEFPLTADNVKAVILDMFGAGTDTSSATIEWAISELIRCPRAMEKVQTELRQALNGKERIQEEDLQELNYLKLVIKETLRLHPPLPLVMPRECREPCVLGGYDIPSKTKLIVNVFAINRDPEYWKDAETFMPERFENSPITVMGSEYEYLPFGAGRRMCPGAALGLANVELPLAHILYYFNWKLPNGKTFEDLDMTESFGATVQRKTELLLVPTDFQTLTAST

SEQ ID NO: 5 - DNA, 2142 bp, Nicotiana sylvestris
atggattctacatcagagaaactttctccttttgattttatggcggcgatctttaaaggtggaaagatattcgatcaactgaactcatcatcagattctggcgactcaagttctcctgcttcgttggcagctctgctgatggagaacaaagatttaatgatgatgttgacaacctcggttgctgtcttgatcggatgtgcagttgtcttgatgtggcggcgctcctcaacttctgctaagaaggtggtagagccgcccaagttggtggttcctaagtcggttattgaacctgaagaaattgatgatggcaagaagaaagttaccatcctttttggtacccagactggtactgctgaaggctttgctaaggcacttgccgaggaagccaaggccagatatgataaggctacctttaaagtgattgatatggatgattatgcggctgatgatgatgactatgaagagaaattgaagaaagaaacattggcattctttttcttggccacatatggagatggtgagccaactgataatgctgccagattctataaatggtttgtcgagggaaaagagaggggtgactactttaaaaatcttcagtatggagtatttggccttggtaacagacagtacgagcatttcaacaagattgcaaaagtggtggatgaccttctcgctgagcaaggcgggcagaggcttgttcctgtgggtcttggagatgatgaccaatgcattgaagatgattttgctgcatggcgtgaattagtgtggcctgaattggataagtttcttctggatggggatgatgcaactgctgcaactccatataccgctgcagttttggaatatagggttgttacccatgaaaagcctaacaacgacttgagtaacacaaatggtcatgcaaatggacatgcaatcattgatgctcaacatccctgcatagctaatgttgctgtgaagaaggagcttcatactcctgcttctgatcgttcttgcactcatttggagtttgacatttctggaactggagttgtttatgaaactggtgatcatgtcggtgtgtactgtgaaaatttgattgaaaccgtagaggaagctgaaaggttactaaacatatcacctgacactttcttttccattcacactgataaagaagatggcacaccactgggcggaagctcattgccgtctcccttccctccttgcactttaagaacagcattgactctgtatgctgatcttttgagttctcctaaaaagtctgctttacttgctttagcggcatgtgcttctgatccaaatgaagctaatcgattaagaaatcttgcatcaccggctggaaaggaagaatatgctcagtggatggttgcaagtcagagaagccttcttgaagtcatggctgaatttccttcagccaagccttcacttggggttttctttgcttctgttgctcctcgcctacaaccgagattctactctatctcatcatctcataggatggcgccatctaggattcatgttacttgtgcactggtttacgacaaaatgccaaccggacgagttcacaagggtgtctgctcaacatggatgaagaatgctgttcctctagaagaaagcctttcctgcagtacagcacctatttttgttcggcaatcaaacttcaaactgccagctgataacaaggttccaatcataatgatcggccctggtactgggttggcaccattcaggggtttcctccaggaaagattagcttttaagaaagaaggagctgagcttggtcctgcagtgttattttttggatgcaggaaccgccaaatggactacatctatcaagaagagttggacaatttccttgaggccggtgcactttctgagctagtagttgctttctctcgtgaaggacctaacaaagaatacgtgcaacataaaatgtcagagaaggctgcggatatctggaacatgatttctcagggaggatacgtatatgtctgcggtgatgcaaaaggcatggctagggacgttcatcgggctcttcacactattgcccaggatcagggatcgctcgacagctccaaggctgaggccttggtgaagaacttgcaaataactggaagatatctgcgtgatgtgtggtga

SEQ ID NO: 6 - protein, 713 aa, Nicotiana sylvestris
MDSTSEKLSPFDFMAAIFKGGKIFDQLNSSSDSGDSSSPASLAALLMENKDLMMMLTTSVAVLIGCAVVLMWRRSSTSAKKVVEPPKLVVPKSVIEPEEIDDGKKKVTILFGTQTGTAEGFAKALAEEAKARYDKATFKVIDMDDYAADDDDYEEKLKKETLAFFFLATYGDGEPTDNAARFYKWFVEGKERGDYFKNLQYGVFGLGNRQYEHFNKIAKVVDDLLAEQGGQRLVPVGLGDDDQCIEDDFAAWRELVWPELDKFLLDGDDATAATPYTAAVLEYRVVTHEKPNNDLSNTNGHANGHAIIDAQHPCIANVAVKKELHTPASDRSCTHLEFDISGTGVVYETGDHVGVYCENLIETVEEAERLLNISPDTFFSIHTDKEDGTPLGGSSLPSPFPPCTLRTALTLYADLLSSPKKSALLALAACASDPNEANRLRNLASPAGKEEYAQWMVASQRSLLEVMAEFPSAKPSLGVFFASVAPRLQPRFYSISSSHRMAPSRIHVTCALVYDKMPTGRVHKGVCSTWMKNAVPLEESLSCSTAPIFVRQSNFKLPADNKVPIIMIGPGTGLAPFRGFLQERLAFKKEGAELGPAVLFFGCRNRQMDYIYQEELDNFLEAGALSELVVAFSREGPNKEYVQHKMSEKAADIWNMISQGGYVYVCGDAKGMARDVHRALHTIAQDQGSLDSSKAEALVKNLQITGRYLRDVW

SEQ ID NO: 7 - DNA, 2079 bp, Arabidopsis thaliana
atgacttctgcactttatgcctccgatcttttcaaacaattgaaaagtatcatgggaacggattctttgtccgatgatgttgtattagttattgctacaacttctctggcactggttgctggtttcgttgtcttattgtggaaaaagaccacggcagatcgttccggcgagctaaagccactaatgatccctaagtctctgatggcgaaagatgaggatgatgacttagatctaggttctggaaaaacgagagtctctatcttcttcggcacacaaaccggaacagccgaaggattcgctaaagcactttcagaagagatcaaagcaagatacgaaaaggcggctgtaaaagtaatcgatttggatgattacgctgccgatgatgaccaatatgaggaaaagttgaaaaaggaaacattggctttcttttgtgtagccacgtatggtgatggtgaaccaaccgataacgccgcaagattctacaagtggtttactgaagagaacgaaagagatatcaagttgcagcaacttgcttacggcgtttttgccttaggtaacagacaatacgagcactttaacaagataggtattgtcttagatgaagagttatgcaaaaagggtgcgaagagattgattgaagtcggtttaggagatgatgatcaatctatcgaggatgactttaatgcatggaaggaatctttgtggtctgaattagataagttacttaaggacgaagatgataaatccgttgccactccatacacagccgtcattccagaatatagagtagttactcatgatccaagattcacaacacagaaatcaatggaaagtaatgtggctaatggtaatactaccatcgatattcatcatccatgtagagtagacgttgcagttcaaaaggaattgcacactcatgaatcagacagatcttgcatacatcttgaatttgatatatcacgtactggtatcacttacgaaacaggtgatcacgtgggtgtctacgctgaaaaccatgttgaaattgtagaggaagctggaaagttgttgggccatagtttagatcttgttttctcaattcatgccgataaagaggatggctcaccactagaaagtgcagtgcctccaccatttccaggaccatgcaccctaggtaccggtttagctcgttacgcggatctgttaaatcctccacgtaaatcagctctagtggccttggctgcgtacgccacagaaccttctgaggcagaaaaactgaaacatctaacttcaccagatggtaaggatgaatactcacaatggatagtagctagtcaacgttctttactagaagttatggctgctttcccatccgctaaacctcctttgggtgttttcttcgccgcaatagcgcctagactgcaaccaagatactattcaatttcatcctcacctagactggcaccatcaagagttcatgtcacatccgctttagtgtacggtccaactcctactggtagaatccataagggcgtttgttcaacatggatgaaaaacgcggttccagcagagaagtctcacgaatgttctggtgctccaatctttatcagagcctccaacttcaaactgccttccaatccttctactcctattgtcatggtcggtcctggtacaggtcttgctccattcagaggtttcttacaagagagaatggccttaaaggaggatggtgaagagttgggatcttctttgttgtttttcggctgtagaaacagacaaatggatttcatctacgaagatgaactgaataactttgtagatcaaggagttatttcagagttgataatggctttttctagagaaggtgctcagaaggagtacgtccaacacaaaatgatggaaaaggccgcacaagtttgggacttaatcaaagaggaaggctatctatatgtctgtggtgatgcaaagggtatggcaagagatgttcacagaacacttcatactatagtccaggaacaggaaggcgttagttcttctgaagcggaagcaattgtgaaaaagttacaaacagagggaagatacttgagagatgtgtggtaa

SEQ ID NO: 8 - protein, 692 aa, Arabidopsis thaliana [sequence omitted]

SEQ ID NO: 9 - DNA, 1470 bp, Arabidopsis thaliana
atgggatctcagatcattcataactcacaaaaaccacatgtagtttgtgttccatatccggctcaaggccacatcaaccctatgatgagagtggctaaactcctccacgccagaggcttctacgtcaccttcgtcaacaccgtctacaaccacaatcgtttccttcgttctcgtgggtccaatgccctagatggacttccttcgttccgatttgagtccattgctgacggtctaccagagacagacatggatgccacgcaggacatcacagctctttgcgagtccaccatgaagaactgtctcgctccgttcagagagcttctccagcggatcaacgctggagataatgttcctccggtaagctgtattgtatctgacggttgtatgagctttactcttgatgttgcggaggagcttggagtcccggaggttcttttttggacaaccagtggctgtgcgttcctggcttatctacacttttatctcttcatcgagaagggcttatgtccgctaaaagatgagagttacttgacgaaggagtacttagaagacacggttatagattttataccaaccatgaagaatgtgaaactaaaggatattcctagcttcatacgtaccactaatcctgatgatgttatgattagtttcgccctccgcgagaccgagcgagccaaacgtgcttctgctatcattctaaacacatttgatgaccttgagcatgatgttgttcatgctatgcaatctatcttacctccggtttattcagttggaccgcttcatctcttagcaaaccgggagattgaagaaggtagtgagattggaatgatgagttcgaatttatggaaagaggagatggagtgtttggattggcttgatactaagactcaaaatagtgtcatttatatcaactttgggagcataacggttttgagtgtgaagcagcttgtggagtttgcttggggtttggcgggaagtgggaaagagtttttatgggtgatccggccagatttagtagcgggagaggaggctatggttccgccggactttttaatggagactaaagaccgcagtatgctagcgagttggtgtcctcaagagaaagtactttctcatcctgctattggagggtttttgacgcattgcgggtggaactcgatattggaaagtctttcgtgtggagttccgatggtgtgttggccattttttgctgaccagcaaatgaattgtaagttttgttgtgacgagtgggatgttgggattgagataggtggagatgtgaagagagaggaagttgaggcggtggttagagagctcatggatggagagaagggaaagaaaatgagagaaaaggcggtagagtggcagcgcttagccgagaaagcgacggaacataaacttggttcttccgttatgaattttgagacggttgttagcaagtttcttttgggacaaaaatcacaggactag

SEQ ID NO: 10 - protein, 489 aa, Arabidopsis thaliana
MGSQIIHNSQKPHVVCVPYPAQGHINPMMRVAKLLHARGFYVTFVNTVYNHNRFLRSRGSNALDGLPSFRFESIADGLPETDMDATQDITALCESTMKNCLAPFRELLQRINAGDNVPPVSCIVSDGCMSFTLDVAEELGVPEVLFWTTSGCAFLAYLHFYLFIEKGLCPLKDESYLTKEYLEDTVIDFIPTMKNVKLKDIPSFIRTTNPDDVMISFALRETERAKRASAIILNTFDDLEHDVVHAMQSILPPVYSVGPLHLLANREIEEGSEIGMMSSNLWKEEMECLDWLDTKTQNSVIYINFGSITVLSVKQLVEFAWGLAGSGKEFLWVIRPDLVAGEEAMVPPDFLMETKDRSMLASWCPQEKVLSHPAIGGFLTHCGWNSILESLSCGVPMVCWPFFADQQMNCKFCCDEWDVGIEIGGDVKREEVEAVVRELMDGEKGKKMREKAVEWQRLAEKATEHKLGSSVMNFETVVSKFLLGQKSQD

SEQ ID NO: 11 - DNA, 1362 bp, Arabidopsis thaliana
atggaagaactaggagtgaagagaaggatagtattggttccagttccagcacaaggtcatgtaactccgattatgcaactcgggaaggctctttactccaagggcttctccatcactgttgttctcacacagtataatcgagttagctcatccaaggacttctctgattttcatttcctcaccatcccaggcagcttgaccgagtctgatctcaaaaaccttggaccattcaagtttctcttcaagctcaatcaaatttgcgaggcaagcttcaagcaatgtattggtcaactattgcaggagcaaggtaatgatatcgcttgtgtcgtctacgatgagtacatgtacttctcccaagctgcagttaaagagtttcaacttcctagcgtcctcttcagcacgacaagtgctactgcctttgtctgtcgctctgttttgtctagagtcaacgcagagtcattcttgcttgacatgaaagatcccaaagtgtcagacaaggaatttccagggttgcatccgctaaggtacaaggacctgccaacttcagcatttgggccattagagagtatactcaaggtttacagtgagactgtcaacattcgaacagcttcggcagttatcatcaactcaacaagctgtctagagagctcatctttggcatggttacaaaaacaactgcaagttccagtgtatcctataggcccacttcacattgcagcttcagcgccttctagtttacttgaagaggacaggagttgccttgagtggttgaacaagcaaaaaataggctcagtgatttacataagtttgggaagcttggctctaatggaaactaaagacatgttggagatggcttggggtttacgtaatagcaaccaacctttcttatgggtgatccgaccgggttctattcccggctcggaatggacagagtctttaccggaggaattcagtaggttggtttcagaaagaggttacattgtgaaatgggcaccacagatagaagttctcagacatcctgcagtgggagggttttggagtcactgcggatggaactcgaccctagagagcatcggggaaggagttccgatgatctgtaggccttttacgggagatcagaaagtcaatgcgaggtacttagagagagtttggagaattggggttcaattggaaggagagctggataaaggaacagtggagagagctgtagagagattgattatggatgaagaaggagcagaaatgaggaagagagttatcaacttgaaagagaagcttcaagcctctgtcaagagtagaggttcctcattcagctcattagacaactttgtcaattccttaaaaatgatgaatttcatgtag

SEQ ID NO: 12 - protein, 453 aa, Arabidopsis thaliana
MEELGVKRRIVLVPVPAQGHVTPIMQLGKALYSKGFSITVVLTQYNRVSSSKDFSDFHFLTIPGSLTESDLKNLGPFKFLFKLNQICEASFKQCIGQLLQEQGNDIACVVYDEYMYFSQAAVKEFQLPSVLFSTTSATAFVCRSVLSRVNAESFLLDMKDPKVSDKEFPGLHPLRYKDLPTSAFGPLESILKVYSETVNIRTASAVIINSTSCLESSSLAWLQKQLQVPVYPIGPLHIAASAPSSLLEEDRSCLEWLNKQKIGSVIYISLGSLALMETKDMLEMAWGLRNSNQPFLWVIRPGSIPGSEWTESLPEEFSRLVSERGYIVKWAPQIEVLRHPAVGGFWSHCGWNSTLESIGEGVPMICRPFTGDQKVNARYLERVWRIGVQLEGELDKGTVERAVERLIMDEEGAEMRKRVINLKEKLQASVKSRGSSFSSLDNFVNSLKMMNFM

SEQ ID NO: 13 - DNA, 1488 bp, Stevia rebaudiana
atgtcgccaaaaatggtggcaccaccaaccaaccttcattttgttttgtttcctcttatggctcaaggccatctggtacccatggtcgacatcgctcgaatcttagcccaacgtggtgcaacggtcaccataatcaccacaccctaccatgccaaccgggtcagaccggttatctcccgagccatcgcgaccaatctcaagatccagctactcgaactccaactgcggtcaaccgaagccggtttacccgaagggtgcgaaagcttcgaccaacttccgtcattcgagtactggaaaaatatttcaaccgctatcgatttgttacaacaacccgctgaagatttgctccgagaactttcaccaccacccgattgcatcatatcggactttttgttcccgtggaccaccgatgtggctcgacggttaaacatcccccggctcgtgttcaatggaccgggctgcttttatctcttgtgcatccatgttgcgatcacttccaacattttgggagagaatgaaccggtcagtagtaataccgagcgcgttgtgctgcccggtttacctgaccggatcgaagtcactaaacttcagatcgtcggttcgtcgagaccagccaacgtagacgaaatgggctcgtggcttcgagccgtagaagctgagaaagcttcattcgggatagtggttaatactttcgaagagcttgaaccggagtacgttgaagaatacaaaacggttaaagataagaagatgtggtgtatcggcccggtttcgttatgcaacaaaaccgggccggatttagccgagcgaggaaacaaagctgcaataaccgaacacaactgcttaaaatggctcgatgagagaaaactggggtccgtgttatacgtttgtttaggtagccttgcacgcatttctgccgcacaagcaatcgagctcgggttaggactcgagtccataaaccgtccctttatatggtgcgtaagaaacgaaaccgatgagctcaaaacatggtttttggatgggtttgaagaaagggttagagatcgcgggttgatcgttcatggttgggcgccacaggttttgatactgtcgcacccaaccattggcggtttcttaacccattgcggttggaactcgactattgaatcgattaccgcgggtgttccaatgatcacgtggccattttttgcggaccagtttttgaatgaagcttttatagttgaagttttgaagattggagttaggattggtgttgagagggcttgtttgtttggggaagaagataaggttggagtgttggtgaagaaggaggatgtgaagaaggctgttgaatgcttgatggatgaagatgaagatggtgatcagagaagaaagagggtgattgagcttgcaaaaatggcgaagattgcaatggcggaaggtggatcttcttatgaaaatgtatcgtcgttgattcgagatgtgactgaaacagttagagcaccacattag

SEQ ID NO: 14 - protein, 495 aa, Stevia rebaudiana
MSPKMVAPPTNLHFVLFPLMAQGHLVPMVDIARILAQRGATVTIITTPYHANRVRPVISRAIATNLKIQLLELQLRSTEAGLPEGCESFDQLPSFEYWKNISTAIDLLQQPAEDLLRELSPPPDCIISDFLFPWTTDVARRLNIPRLVFNGPGCFYLLCIHVAITSNILGENEPVSSNTERVVLPGLPDRIEVTKLQIVGSSRPANVDEMGSWLRAVEAEKASFGIVVNTFEELEPEYVEEYKTVKDKKMWCIGPVSLCNKTGPDLAERGNKAAITEHNCLKWLDERKLGSVLYVCLGSLARISAAQAIELGLGLESINRPFIWCVRNETDELKTWFLDGFEERVRDRGLIVHGWAPQVLILSHPTIGGFLTHCGWNSTIESITAGVPMITWPFFADQFLNEAFIVEVLKIGVRIGVERACLFGEEDKVGVLVKKEDVKKAVECLMDEDEDGDQRRKRVIELAKMAKIAMAEGGSSYENVSSLIRDVTETVRAPH

SEQ ID NO: 15 - DNA, 1665 bp, Arabidopsis thaliana
aaaaacacataacttgagtattcctcattgagtcgttgcaaacggtatggctacggaaaaaacccaccaatttcatccttctcttcactttgtcctcttccctttcatggctcaaggccacatgattcccatgattgatattgcaagactcttggctcagcgtggtgtgaccataacaattgtcacgacacctcacaacgcagcaaggtttaagaatgtcctaaaccgagcgatcgagtctggcttggccatcaacatactgcatgtgaagtttccatatcaagagtttggtttgccagaaggaaaagagaatatagattcgttagactcaacggagttgatggtacctttcttcaaagcggtgaacttgcttgaagatccggtcatgaagctcatggaagagatgaaacctagacctagctgtctaatttctgattggtgtttgccttatacaagcataatcgccaagaacttcaatataccaaagatagttttccacggcatgggttgctttaatcttttgtgtatgcatgttctacgcagaaacttagagatcctagagaatgtaaagtcggatgaagagtatttcttggttcctagttttcctgatagagttgaatttacaaagcttcaacttcctgtgaaagcaaatgcaagtggagattggaaagagataatggatgaaatggtaaaagcagaatacacatcctatggtgtgatcgtcaacacatttcaggagttggagccaccttatgtcaaagactacaaagaggcaatggatggaaaagtatggtccattggacccgtttccttgtgtaacaaggcaggtgcagacaaagctgagaggggaagcaaggccgccattgatcaagatgagtgtcttcaatggcttgattctaaagaagaaggttcggtgctctatgtttgccttggaagtatatgtaatcttcctttgtctcagctcaaggagctggggctaggccttgaggaatctcgaagatcttttatttgggtcataagaggttcggaaaagtataaagaactatttgagtggatgttggagagcggttttgaagaaagaatcaaagagagaggacttctcattaaagggtgggcacctcaagtccttatcctttcacatccttccgttggaggattcctgacacactgtggatggaactcgactctcgaaggaatcacctcaggcattccactgatcacttggccgctgtttggagaccaattctgcaaccaaaaactggtcgttcaagtactaaaagccggtgtaagtgccggggttgaagaagtcatgaaatggggagaagaagataaaataggagtgttagtggataaagaaggagtgaaaaaggctgtggaagaattgatgggtgatagtgatgatgcaaaagagaggagaagaagagtcaaagagcttggagaattagctcacaaagctgtggaaaaaggaggctcttctcattctaacatcacactcttgctacaagacataatgcaactagcacaattcaagaattgagcatatgtcattttatgttcatagaaatttaaacattaaacagtttttgatttctatattggagaaatttaaatcagagcctttgttaaacacgtggataatgaatcagaagaagataaatcagtgat

SEQ ID NO: 16 - protein, 496 aa, Arabidopsis thaliana
MATEKTHQFHPSLHFVLFPFMAQGHMIPMIDIARLLAQRGVTITIVTTPHNAARFKNVLNRAIESGLAINILHVKFPYQEFGLPEGKENIDSLDSTELMVPFFKAVNLLEDPVMKLMEEMKPRPSCLISDWCLPYTSIIAKNFNIPKIVFHGMGCFNLLCMHVLRRNLEILENVKSDEEYFLVPSFPDRVEFTKLQLPVKANASGDWKEIMDEMVKAEYTSYGVIVNTFQELEPPYVKDYKEAMDGKVWSIGPVSLCNKAGADKAERGSKAAIDQDECLQWLDSKEEGSVLYVCLGSICNLPLSQLKELGLGLEESRRSFIWVIRGSEKYKELFEWMLESGFEERIKERGLLIKGWAPQVLILSHPSVGGFLTHCGWNSTLEGITSGIPLITWPLFGDQFCNQKLVVQVLKAGVSAGVEEVMKWGEEDKIGVLVDKEGVKKAVEELMGDSDDAKERRRRVKELGELAHKAVEKGGSSHSNITLLLQDIMQLAQFKN

SEQ ID NO: 17 - DNA, 1824 bp, Arabidopsis thaliana
cctttggcacgtttctcaatatagattgctgcaccagaaaaagacaattttttctgtttgaagcctcttttgacatgaagcgaaatgcggcaagagaaccaagaagacaatagcatttttgcttttctttggttaccactctctaacaagataaagaaatgtaacaccatccaccacatgattcacatgttgtgacgaagagagccaacaaaacgttcgagagtcagatactttatatatcggttaccgaagtgatcgaaaccgcgaaccacgaagctgagcaggatacttttaattagtagttcatgcaggttttgggaatggaggaaaagcctgcaaggagaagcgtagtgttggttccatttccagcacaaggacatatatctccaatgatgcaacttgccaaaacccttcacttaaagggtttctcgatcacagttgttcagactaagttcaattactttagcccttcagatgacttcactcatgattttcagttcgtcaccattccagaaagcttaccagagtctgatttcaagaatctcggaccaatacagtttctgtttaagctcaacaaagagtgtaaggtgagcttcaaggactgtttgggtcagttggtgctgcaacaaagtaatgagatctcatgtgtcatctacgatgagttcatgtactttgctgaagctgcagccaaagagtgtaagcttccaaacatcattttcagcacaacaagtgccacggctttcgcttgccgctctgtatttgacaaactatatgcaaacaatgtccaagctcccttgaaagaaactaaaggacaacaagaagagctagttccggagttttatcccttgagatataaagactttccagtttcacggtttgcatcattagagagcataatggaggtgtataggaatacagttgacaaacggacagcttcctcggtgataatcaacactgcgagctgtctagagagctcatctctgtcttttctgcaacaacaacagctacaaattccagtgtatcctataggccctcttcacatggtggcctcagctcctacaagtctgcttgaagagaacaagagctgcatcgaatggttgaacaaacaaaaggtaaactcggtgatatacataagcatgggaagcatagctttaatggaaatcaacgagataatggaagtcgcgtcaggattggctgctagcaaccaacacttcttatgggtgatccgaccagggtcaatacctggttccgagtggatagagtccatgcctgaagagtttagtaagatggttttggaccgaggttacattgtgaaatgggctccacagaaggaagtactttctcatcctgcagtaggagggttttggagccattgtggatggaactcgacactagaaagcatcggccaaggagttccaatgatctgcaggccattttcgggtgatcaaaaggtgaacgctagatacttggagtgtgtatggaaaattgggattcaagtggagggtgagctagacagaggagtggtcgagagagctgtgaagaggttaatggttgacgaagaaggagaggagatgaggaagagagctttcagtttaaaagagcaacttagagcctctgttaaaagtggaggctcttcacacaactcgctagaagagtttgtacacttcataaggactctatgaagacagttgtgtctactaaaacacacaaagcatcaagattcctttttcaaccattccaagtttgtttgcatagagtttatctgaacatcacttaaaaactcacctgcagaataacaaactataaaaattaatactttaccgc

SEQ ID NO: 18 - protein, 458 aa, Arabidopsis thaliana
MQVLGMEEKPARRSVVLVPFPAQGHISPMMQLAKTLHLKGFSITVVQTKFNYFSPSDDFTHDFQFVTIPESLPESDFKNLGPIQFLFKLNKECKVSFKDCLGQLVLQQSNEISCVIYDEFMYFAEAAAKECKLPNIIFSTTSATAFACRSVFDKLYANNVQAPLKETKGQQEELVPEFYPLRYKDFPVSRFASLESIMEVYRNTVDKRTASSVIINTASCLESSSLSFLQQQQLQIPVYPIGPLHMVASAPTSLLEENKSCIEWLNKQKVNSVIYISMGSIALMEINEIMEVASGLAASNQHFLWVIRPGSIPGSEWIESMPEEFSKMVLDRGYIVKWAPQKEVLSHPAVGGFWSHCGWNSTLESIGQGVPMICRPFSGDQKVNARYLECVWKIGIQVEGELDRGVVERAVKRLMVDEEGEEMRKRAFSLKEQLRASVKSGGSSHNSLEEFVHFIRTL

SEQ ID NO: 19 - DNA, 1698 bp, Eryngium glaciale
atgtctcttaatgtacttagtacgtcaggttcagctccaacaaccaaatcatctgagattactcgtaggtccgctaattatcatcctagtttatggggagacaagttcctcgaatattcgagcccagatcacctgaaaaatgattcattcacagaaaagaaacatgaacaactcaaagaagaggtgaagaagatgctagtagaaacggttcaaaagcctcaacaacagctgaatctgatcaacgaaatacaacgactaggtttatcatacctttttgaacccgaaattgaggctgcattgcaggaaatcagtgttacctatgatgaattttgttgtagtacagacgctgatgaccttcacaatgttgctctctctttccgaatacttagagaacatggacataatgtatcttctgatgtgtttcagaaattcatggatagcaatgggaagttgaaagactacttggttaatgatgctagaggactgttaagcttgtacgaagcaacacattttcgggttcataatgatgataaacttgaagagttgctgtcagtaacaacctctcgtcttgagcatctcaaatcccacgtgaagtaccctcttgaggacgaaatcagtagagcacttaagcatcccctccataaagaactaaatcgactaggagcgagatattacatatccatttacgaaaaatttgattcacacaataaattgcttttggagtttgcaaaactagattttaaccgactgcagaaaatgtatcaacatgagctagcccaccttacaaggtggtggaaagatttagattttacaaacaaacttccatttgcaagagatagaattgttgagggttacttttggatcttaggaatgtactttgagccagaacgtaaggatgtcagggaattcttgaacagagtatttgcacttattacagtagttgatgacacgtatgatgtgtatggtaccttcaaagaacttctactgttcactgatgcaattgaaagatggggaactagtgatttggatcagctaccgggatatatgagaattatttatcaagctctcatggatgtttataatcaaatggaggaaaagttgtcaatgaaagctgattgtccaacataccgtcttgagtttgcaatagaaacagttaaagccatgttcagatcatacctcgaagaagctagatggtccaaagaacattatatcccatcgatggaagagtatatgaccgtggcactggtatcggttggctacaaaaccatattaactaattcctttgttggaatgggggatattgcaacacgggaagtttttgagtgggtgttcaatagtccattgattattagagcttccgacttaattgccagattgggagatgatattggaggccatgaggaggagcagaagaaaggagacgcagccactgctatcgagtgttacataaaagagaatcatgtaacaaagcatgaagcttatgatgaatttcagaaacaaattgataatgcttggaaggatttgaataaggaagctctacgtccatttcctgttccaatgactttcatcacaagagttgttcattttacgcgcgccatacatgttatttatgccgactttagtgatggttacacacgttcagacaaggcgatcagaggttacataacttcactgctcgtggatcctattcctttgtaa

SEQ ID NO: 20 - protein, 565 aa, Eryngium glaciale
MSLNVLSTSGSAPTTKSSEITRRSANYHPSLWGDKFLEYSSPDHLKNDSFTEKKHEQLKEEVKKMLVETVQKPQQQLNLINEIQRLGLSYLFEPEIEAALQEISVTYDEFCCSTDADDLHNVALSFRILREHGHNVSSDVFQKFMDSNGKLKDYLVNDARGLLSLYEATHFRVHNDDKLEELLSVTTSRLEHLKSHVKYPLEDEISRALKHPLHKELNRLGARYYISIYEKFDSHNKLLLEFAKLDFNRLQKMYQHELAHLTRWWKDLDFTNKLPFARDRIVEGYFWILGMYFEPERKDVREFLNRVFALITVVDDTYDVYGTFKELLLFTDAIERWGTSDLDQLPGYMRIIYQALMDVYNQMEEKLSMKADCPTYRLEFAIETVKAMFRSYLEEARWSKEHYIPSMEEYMTVALVSVGYKTILTNSFVGMGDIATREVFEWVFNSPLIIRASDLIARLGDDIGGHEEEQKKGDAATAIECYIKENHVTKHEAYDEFQKQIDNAWKDLNKEALRPFPVPMTFITRVVHFTRAIHVIYADFSDGYTRSDKAIRGYITSLLVDPIPL

SEQ ID NO: 21 - DNA, 1347 bp, Saccharomyces cerevisiae
atgctttcgcttaaaacgttactgtgtacgttgttgactgtgtcatcagtactcgctaccccagtccctgcaagagacccttcttccattcaatttgttcatgaggagaacaagaaaagatactacgattatgaccacggttccctcggagaaccaatccgtggtgtcaacattggtggttggttacttcttgaaccatacattactccatctttgttcgaggctttccgtacaaatgatgacaacgacgaaggaattcctgtcgacgaatatcacttctgtcaatatttaggtaaggatttggctaaaagccgtttacagagccattggtctactttctaccaagaacaagatttcgctaatattgcttcccaaggtttcaaccttgtcagaattcctatcggttactgggctttccaaactttggacgatgatccttatgttagcggcctacaggaatcttacctagaccaagccatcggttgggctagaaacaacagcttgaaagtttgggttgatttgcatggtgccgctggttcgcagaacgggtttgataactctggtttgagagattcatacaagtttttggaagacagcaatttggccgttactacaaatgtcttgaactacatattgaaaaaatactctgcggaggaatacttggacactgttattggtatcgaattgattaatgagccattgggtcctgttctagacatggataaaatgaagaatgactacttggcacctgcttacgaatacttgagaaacaacatcaagagtgaccaagttatcatcatccatgacgctttccaaccatacaattattgggatgacttcatgactgaaaacgatggctactggggtgtcactatcgaccatcatcactaccaagtctttgcttctgatcaattggaaagatccattgatgaacatattaaagtagcttgtgaatggggtaccggagttttgaatgaatcccactggactgtttgtggtgagtttgctgccgctttgactgattgtacaaaatggttgaatagtgttggcttcggcgctagatacgacggttcttgggtcaatggtgaccaaacatcttcttacattggctcttgtgctaacaacgatgatatagcttactggtctgacgaaagaaaggaaaacacaagacgttatgtggaggcacaactagatgcctttgaaatgagagggggttggattatctggtgttacaagacagaatctagtttggaatgggatgctcaaagattgatgttcaatggtttattccctcaaccattgactgacagaaagtatccaaaccaatgtggcacaatttctaactaa

SEQ ID NO: 22 - protein, 448 aa, Saccharomyces cerevisiae
MLSLKTLLCTLLTVSSVLATPVPARDPSSIQFVHEENKKRYYDYDHGSLGEPIRGVNIGGWLLLEPYITPSLFEAFRTNDDNDEGIPVDEYHFCQYLGKDLAKSRLQSHWSTFYQEQDFANIASQGFNLVRIPIGYWAFQTLDDDPYVSGLQESYLDQAIGWARNNSLKVWVDLHGAAGSQNGFDNSGLRDSYKFLEDSNLAVTTNVLNYILKKYSAEEYLDTVIGIELINEPLGPVLDMDKMKNDYLAPAYEYLRNNIKSDQVIIIHDAFQPYNYWDDFMTENDGYWGVTIDHHHYQVFASDQLERSIDEHIKVACEWGTGVLNESHWTVCGEFAAALTDCTKWLNSVGFGARYDGSWVNGDQTSSYIGSCANNDDIAYWSDERKENTRRYVEAQLDAFEMRGGWIIWCYKTESSLEWDAQRLMFNGLFPQPLTDRKYPNQCGTISN